SEQUENCE LISTING (55 sequences)

SEQ ID NO: 1 - protein, 15 aa, Mycobacterium tuberculosis
DEVVAVGAALQAGVL

SEQ ID NO: 2 - protein, 15 aa, Mus musculus
DEAVAIGAAIQGGVL

SEQ ID NO: 3 - protein, 15 aa, Mycobacterium tuberculosis
VLRIVNEPTAAALAY

SEQ ID NO: 4 - protein, 15 aa, Mus musculus
VLRVINEPTAAALAY

SEQ ID NO: 5 - protein, 15 aa, Mus musculus
VLRIINEPTAAAIAY

SEQ ID NO: 6 - protein, 15 aa, Mycobacterium tuberculosis
KPFQSVIADTGISVS

SEQ ID NO: 7 - protein, 15 aa, Mycobacterium tuberculosis
YTAPEISARILMKLK

SEQ ID NO: 8 - protein, 15 aa, Mycobacterium tuberculosis
AEGGSKVPEDTLNKV

SEQ ID NO: 9 - protein, 15 aa, Mycobacterium tuberculosis
AQAASQATGAAHPGG

SEQ ID NO: 10 - protein, 15 aa, Mycobacterium tuberculosis
EGSRTTPSIVAFARN

SEQ ID NO: 11 - protein, 15 aa, Mycobacterium tuberculosis
MQRLREAAEKAKIEL

SEQ ID NO: 12 - protein, 15 aa, Mycobacterium tuberculosis
GGKEPNKGVNPDEVV

SEQ ID NO: 13 - protein, 15 aa, Mycobacterium tuberculosis
LDVTPLSLGIETKGG

SEQ ID NO: 14 - protein, 15 aa, Mycobacterium tuberculosis
ILVFDLGGGTFDVSL

SEQ ID NO: 15 - protein, 15 aa, Mycobacterium tuberculosis
RGIPQIEVTFDIDAN

SEQ ID NO: 16 - protein, 15 aa, Mycobacterium tuberculosis
QIEVTFDIDANGIVH

SEQ ID NO: 17 - protein, 14 aa, Mus musculus
IIANDQGNRTTPSY

SEQ ID NO: 18 - protein, 15 aa, Mus musculus
ITPSYVAFTPEGERL

SEQ ID NO: 19 - protein, 15 aa, Homo sapiens
TPSYVAFTDTERLIG

SEQ ID NO: 20 - protein, 17 aa, Homo sapiens
TPSYVAFTDTERLIGDA

SEQ ID NO: 21 - protein, 13 aa, Mus musculus
DAAKNQLTSNPEN

SEQ ID NO: 22 - protein, 18 aa, Homo sapiens
NPTNTVFDAKRLIGRRFD

SEQ ID NO: 23 - protein, 18 aa, Mus musculus
QDIKFLPFKVVEKKTKPY

SEQ ID NO: 24 - protein, 18 aa, Rattus norvegicus
LNVLRIINEPTAAAIAYG

SEQ ID NO: 25 - protein, 17 aa, Rattus norvegicus
NVLRIINEPTAAAIAYG

SEQ ID NO: 26 - protein, 17 aa, Homo sapiens
NVLRIINEPTAAAIAYG

SEQ ID NO: 27 - protein, 15 aa, Homo sapiens
NVLRIINEPTAAAIA

SEQ ID NO: 28 - protein, 17 aa, Homo sapiens
NVMRIINEPTAAAIAYG

SEQ ID NO: 29 - protein, 16 aa, Homo sapiens
VMRIINEPTAAAIAYG

SEQ ID NO: 30 - protein, 15 aa, Homo sapiens
IINEPTAAAIAYGLD

SEQ ID NO: 31 - protein, 14 aa, Mus musculus
NRMVNHFIAEFKRK

SEQ ID NO: 32 - protein, 14 aa, Mus musculus
RMVNHFIAEFKRKH

SEQ ID NO: 33 - protein, 15 aa, Homo sapiens
VNHFIAEFKRKHKKD

SEQ ID NO: 34 - protein, 14 aa, Homo sapiens
  misc_feature (1)..(1): Xaa can be any naturally occurring amino acid
XDFYTSITRAXFEE

SEQ ID NO: 35 - protein, 17 aa, Mus musculus
EGEDFSETLTRAKFEEL

SEQ ID NO: 36 - protein, 13 aa, Homo sapiens
ADLFRGTLDPVEK

SEQ ID NO: 37 - protein, 18 aa, Rattus rattus
TIPTKQTQTFTTYSDNQP

SEQ ID NO: 38 - protein, 20 aa, Homo sapiens
VPTKKSQIFSTASDNQPTVT

SEQ ID NO: 39 - protein, 13 aa, Homo sapiens
GERAMTKDNNLLG

SEQ ID NO: 40 - protein, 16 aa, Homo sapiens
GERAMTKDNNLLGKFE

SEQ ID NO: 41 - protein, 16 aa, Homo sapiens
GERAMTKDNNLLGRFE

SEQ ID NO: 42 - protein, 17 aa, Homo sapiens
ANGILNVSAVDKSTGKE

SEQ ID NO: 43 - protein, 14 aa, Homo sapiens
GILNVSAVDKSTGK

SEQ ID NO: 44 - protein, 15 aa, Homo sapiens
GILNVSAVDKSTGKE

SEQ ID NO: 45 - protein, 12 aa, Homo sapiens
CNEIINWLDKNQ

SEQ ID NO: 46 - protein, 16 aa, Mus musculus
ISWLDKNQTAEKEEFE

SEQ ID NO: 47 - protein, 20 aa, Mus musculus
YGSGGPPPTGEEDTSEKDEL

SEQ ID NO: 48 - protein, 641 aa, Homo sapiens
MAKAAAIGIDLGTTYSCVGVFQHGKVEIIANDQGNRTTPSYVAFTDTERLIGDAAKNQVALNPQNTVFDAKRLIGRKFGDPVVQSDMKHWPFQVINDGDKPKVQVSYKGETKAFYPEEISSMVLTKMKEIAEAYLGYPVTNAVITVPAYFNDSQRQATKDAGVIAGLNVLRIINEPTAAAIAYGLDRTGKGERNVLIFDLGGGTFDVSILTIDDGIFEVKATAGDTHLGGEDFDNRLVNHFVEEFKRKHKKDISQNKRAVRRLRTACERAKRTLSSSTQASLEIDSLFEGIDFYTSITRARFEELCSDLFRSTLEPVEKALRDAKLDKAQIHDLVLVGGSTRIPKVQKLLQDFFNGRDLNKSINPDEAVAYGAAVQAAILMGDKSENVQDLLLLDVAPLSLGLETAGGVMTALIKRNSTIPTKQTQIFTTYSDNQPGVLIQVYEGERAMTKDNNLLGRFELSGIPPAPRGVPQIEVTFDIDANGILNVTATDKSTGKANKITITNDKGRLSKEEIERMVQEAEKYKAEDEVQRERVSAKNALESYAFNMKSAVEDEGLKGKISEADKKKVLDKCQEVISWLDANTLAEKDEFEHKRKELEQVCNPIISGLYQGAGGPGPGGFGAQGPKGGSGSGPTIEEVD

SEQ ID NO: 49 - protein, 641 aa, Homo sapiens
MATAKGIAIGIDLGTTYSCVGVFQHGKVEIIANDQGNRTTPSYVAFTDTERLIGDAAKNQVAMNPQNTVFDAKRLIGRKFNDPVVQADMKLWPFQVINEGGKPKVLVSYKGENKAFYPEEISSMVLTKLKETAEAFLGHPVTNAVITVPAYFNDSQRQATKDAGVIAGLNVLRIINEPTAAAIAYGLDKGGQGERHVLIFDLGGGTFDVSILTIDDGIFEVKATAGDTHLGGEDFDNRLVSHFVEEFKRKHKKDISQNKRAVRRLRTACERAKRTLSSSTQANLEIDSLYEGIDFYTSITRARFEELCADLFRGTLEPVEKALRDAKMDKAKIHDIVLVGGSTRIPKVQRLLQDYFNGRDLNKSINPDEAVAYGAAVQAAILMGDKSEKVQDLLLLDVAPLSLGLETAGGVMTALIKRNSTIPTKQTQIFTTYSDNQPGVLIQVYEGERAMTKDNNLLGRFDLTGIPPAPRGVPQIEVTFDIDANGILNVTATDKSTGKVNKITITNDKGRLSKEEIERMVLDAEKYKAEDEVQREKIAAKNALESYAFNMKSVVSDEGLKGKISESDKNKILDKCNELLSWLEVNQLAEKDEFDHKRKELEQMCNPIITKLYQGGCTGPACGTGYVPGRPATGPTIEEVD

SEQ ID NO: 50 - protein, 639 aa, Homo sapiens
MSARGPAIGIDLGTTYSCVGVFQHGKVEIIANDQGNRTTPSYVAFTDTERLIGDAAKNQVAMNPTNTIFDAKRLIGRKFEDATVQSDMKHWPFRVVSEGGKPKVQVEYKGETKTFFPEEISSMVLTKMKEIAEAYLGGKVHSAVITVPAYFNDSQRQATKDAGTITGLNVLRIINEPTAAAIAYGLDKKGCAGGEKNVLIFDLGGGTFDVSILTIEDGIFEVKSTAGDTHLGGEDFDNRMVSHLAEEFKRKHKKDIGPNKRAVRRLRTACERAKRTLSSSTQASIEIDSLYEGVDFYTSITRARFEELNADLFRGTLEPVEKALRDAKLDKGQIQEIVLVGGSTRIPKIQKLLQDFFNGKELNKSINPDEAVAYGAAVQAAILIGDKSENVQDLLLLDVTPLSLGIETAGGVMTPLIKRNTTIPTKQTQTFTTYSDNQSSVLVQVYEGERAMTKDNNLLGKFDLTGIPPAPRGVPQIEVTFDIDANGILNVTAADKSTGKENKITITNDKGRLSKDDIDRMVQEAERYKSEDEANRDRVAAKNALESYTYNIKQTVEDEKLRGKISEQDKNKILDKCQEVINWLDRNQMAEKDEYEHKQKELERVCNPIISKLYQGGPGGGSGGGGSGASGGPTIEEVD

SEQ ID NO: 51 - protein, 654 aa, Homo sapiens
MKLSLVAAMLLLLSAARAEEEDKKEDVGTVVGIDLGTTYSCVGVFKNGRVEIIANDQGNRITPSYVAFTPEGERLIGDAAKNQLTSNPENTVFDAKRLIGRTWNDPSVQQDIKFLPFKVVEKKTKPYIQVDIGGGQTKTFAPEEISAMVLTKMKETAEAYLGKKVTHAVVTVPAYFNDAQRQATKDAGTIAGLNVMRIINEPTAAAIAYGLDKREGEKNILVFDLGGGTFDVSLLTIDNGVFEVVATNGDTHLGGEDFDQRVMEHFIKLYKKKTGKDVRKDNRAVQKLRREVEKAKRALSSQHQARIEIESFYEGEDFSETLTRAKFEELNMDLFRSTMKPVQKVLEDSDLKKSDIDEIVLVGGSTRIPKIQQLVKEFFNGKEPSRGINPDEAVAYGAAVQAGVLSGDQDTGDLVLLDVCPLTLGIETVGGVMTKLIPRNTVVPTKKSQIFSTASDNQPTVTIKVYEGERPLTKDNHLLGTFDLTGIPPAPRGVPQIEVTFEIDVNGILRVTAEDKGTGNKNKITITNDQNRLTPEEIERMVNDAEKFAEEDKKLKERIDTRNELESYAYSLKNQIGDKEKLGGKLSSEDKETMEKAVEEKIEWLESHQDADIEDFKAKKKELEEIVQPIISKLYGSAGPPPTGEEDTAEKDEL

SEQ ID NO: 52 - protein, 643 aa, Homo sapiens
MQAPRELAVGIDLGTTYSCVGVFQQGRVEILANDQGNRTTPSYVAFTDTERLVGDAAKSQAALNPHNTVFDAKRLIGRKFADTTVQSDMKHWPFRVVSEGGKPKVRVCYRGEDKTFYPEEISSMVLSKMKETAEAYLGQPVKHAVITVPAYFNDSQRQATKDAGAIAGLNVLRIINEPTAAAIAYGLDRRGAGERNVLIFDLGGGTFDVSVLSIDAGVFEVKATAGDTHLGGEDFDNRLVNHFMEEFRRKHGKDLSGNKRALRRLRTACERAKRTLSSSTQATLEIDSLFEGVDFYTSITRARFEELCSDLFRSTLEPVEKALRDAKLDKAQIHDVVLVGGSTRIPKVQKLLQDFFNGKELNKSINPDEAVAYGAAVQAAVLMGDKCEKVQDLLLLDVAPLSLGLETAGGVMTTLIQRNATIPTKQTQTFTTYSDNQPGVFIQVYEGERAMTKDNNLLGRFELSGIPPAPRGVPQIEVTFDIDANGILSVTATDRSTGKANKITITNDKGRLSKEEVERMVHEAEQYKAEDEAQRDRVAAKNSLEAHVFHVKGSLQEESLRDKIPEEDRRKMQDKCREVLAWLEHNQLAEKEEYEHQKRELEQICRPIFSRLYGGPGVPGGSSCGTQARQGDPSTGPIIEEVD

SEQ ID NO: 53 - protein, 646 aa, Homo sapiens
MSKGPAVGIDLGTTYSCVGVFQHGKVEIIANDQGNRTTPSYVAFTDTERLIGDAAKNQVAMNPTNTVFDAKRLIGRRFDDAVVQSDMKHWPFMVVNDAGRPKVQVEYKGETKSFYPEEVSSMVLTKMKEIAEAYLGKTVTNAVVTVPAYFNDSQRQATKDAGTIAGLNVLRIINEPTAAAIAYGLDKKVGAERNVLIFDLGGGTFDVSILTIEDGIFEVKSTAGDTHLGGEDFDNRMVNHFIAEFKRKHKKDISENKRAVRRLRTACERAKRTLSSSTQASIEIDSLYEGIDFYTSITRARFEELNADLFRGTLDPVEKALRDAKLDKSQIHDIVLVGGSTRIPKIQKLLQDFFNGKELNKSINPDEAVAYGAAVQAAILSGDKSENVQDLLLLDVTPLSLGIETAGGVMTVLIKRNTTIPTKQTQTFTTYSDNQPGVLIQVYEGERAMTKDNNLLGKFELTGIPPAPRGVPQIEVTFDIDANGILNVSAVDKSTGKENKITITNDKGRLSKEDIERMVQEAEKYKAEDEKQRDKVSSKNSLESYAFNMKATVEDEKLQGKINDEDKQKILDKCNEIINWLDKNQTAEKEEFEHQQKELEKVCNPIITKLYQSAGGMPGGMPGGFPGGGAPPSGGASSGPTIEEVD

SEQ ID NO: 54 - protein, 679 aa, Homo sapiens
MISASRAAAARLVGAAASRGPTAARHQDSWNGLSHEAFRLVSRRDYASEAIKGAVVGIDLGTTNSCVAVMEGKQAKVLENAEGARTTPSVVAFTADGERLVGMPAKRQAVTNPNNTFYATKRLIGRRYDDPEVQKDIKNVPFKIVRASNGDAWVEAHGKLYSPSQIGAFVLMKMKETAENYLGHTAKNAVITVPAYFNDSQRQATKDAGQISGLNVLRVINEPTAAALAYGLDKSEDKVIAVYDLGGGTFDISILEIQKGVFEVKSTNGDTFLGGEDFDQALLRHIVKEFKRETGVDLTKDNMALQRVREAAEKAKCELSSSVQTDINLPYLTMDSSGPKHLNMKLTRAQFEGIVTDLIRRTIAPCQKAMQDAEVSKSDIGEVILVGGMTRMPKVQQTVQDLFGRAPSKAVNPDEAVAIGAAIQGGVLAGDVTDVLLLDVTPLSLGIETLGGVFTKLINRNTTIPTKKSQVFSTAADGQTQVEIKVCQGEREMAGDNKLLGQFTLIGIPPAPRGVPQIEVTFDIDANGIVHVSAKDKGTGREQQIVIQSSGGLSKDDIENMVKNAEKYAEEDRRKKERVEAVNMAEGIIHDTETKMEEFKDQLPADECNKLKEEISKMRELLARKDSETGENIRQAASSLQQASLKLFEMAYKKMASEREGSGSSGTGEQKEDQKEEKQ

SEQ ID NO: 55 - protein, 625 aa, Mycobacterium tuberculosis
MARAVGIDLGTTNSVVSVLEGGDPVVVANSEGSRTTPSIVAFARNGEVLVGQPAKNQAVTNVDRTVRSVKRHMGSDWSIEIDGKKYTAPEISARILMKLKRDAEAYLGEDITDAVITTPAYFNDAQRQATKDAGQIAGLNVLRIVNEPTAAALAYGLDKGEKEQRILVFDLGGGTFDVSLLEIGEGVVEVRATSGDNHLGGDDWDQRVVDWLVDKFKGTSGIDLTKDKMAMQRLREAAEKAKIELSSSQSTSINLPYITVDADKNPLFLDEQLTRAEFQRITQDLLDRTRKPFQSVIADTGISVSEIDHVVLVGGSTRMPAVTDLVKELTGGKEPNKGVNPDEVVAVGAALQAGVLKGEVKDVLLLDVTPLSLGIETKGGVMTRLIERNTTIPTKRSETFTTADDNQPSVQIQVYQGEREIAAHNKLLGSFELTGIPPAPRGIPQIEVTFDIDANGIVHVTAKDKGTGKENTIRIQEGSGLSKEDIDRMIKDAEAHAEEDRKRREEADVRNQAETLVYQTEKFVKEQREAEGGSKVPEDTLNKVDAAVAEAKAALGGSDISAIKSAMEKLGQESQALGQAIYEAAQAASQATGAAHPGGEPGGAHPGSADDVVDAEVVDDGREAK

(19) United States
(12) Patent Application Publication
(10) Pub. No.: US 2009/0118173 A1
(43) Pub. Date: May 7, 2009

(54) TREATMENT AND PREVENTION OF INFLAMMATORY BOWEL DISEASES

(75) Inventors: Willem van Eden, Bilthoven (NL); Ruurd van der Zee, Utrecht (NL); Helena Tlaskalova-Hogenova, Prague (CZ); Miloslav Kverka, Prague (CZ)

Correspondence Address: FOLEY AND LARDNER LLP;SUITE 500, 3000 K STREET NW, WASHINGTON, DC, 20007, US

(73) Assignee: Universiteit Utrecht UU Holding B.V.

(21) Appl. No.: 11/774,405

(22) Filed: Jul. 6, 2007

Related U.S. Application Data
(63) Continuation-in-part of application No. 11/626,152, filed on Jan. 23, 2007.
(60) Provisional application No. 60/761,339, filed on Jan. 24, 2006.

(51) Int. Cl. A61K 38/16; G01N 33/53; A61P 1/06

(52) U.S. Cl. 514/12; 435/7.24

(57) ABSTRACT

The present invention relates to the use of heat shock proteins, or fragments thereof, for the treatment and/or prevention of Inflammatory Bowel Diseases. Preferably bacterial and/or mammalian heat shock proteins belonging to the HSP70 families are used.

### PRIORITY

This application is a continuation-in-part of U.S. patent application Ser. No. 11/626,152, filed Jan. 23, 2007, which claims the benefit of U.S. application No. 60/761,339, filed Jan. 24, 2007, the disclosures of which are both incorporated herein in their entirety by reference.

### FIELD OF THE INVENTION

The present invention relates to the treatment and/or prevention of Inflammatory Bowel Diseases (IBD), including Crohn's disease, granulomatous colitis, ulcerative colitis, lymphocyte colitis, collagenous colitis and Coeliac disease. Especially, the use of human MHC class II eluted fragments of heat shock protein 70 family members and their extended sequences (such as longer fragments and/or a panel of peptides), for the preparation of compositions for the treatment of one or more IBDs is provided, as are methods for therapeutic and/or prophylactic treatment of one or more IBDs. Also, the use of full length heat shock protein 70 family members for the therapeutic and/or prophylactic treatment of one or more IBDs is provided herein. The invention relates especially to the use of heat shock 70 proteins and/or peptides for treatment of non-autoimmune diseases, such as the above IBDs. Further, a method for identifying and selecting peptides of heat shock protein 70 (HSP70) family members, suitable for the treatment and/or prevention of one or more IBDs, is provided.

### BACKGROUND OF THE INVENTION

Heat shock proteins (HSP) have shown to be critical to protect against type 1 diabetes mellitus and rheumatoid arthritis, both of which are prevalent chronic degenerative autoimmune diseases.

The criticality was based on the following findings:
1. Peptides of HSP's can be used as therapeutic agents to prevent or arrest the inflammatory damage in both experimental autoimmune arthritis and in experimental autoimmune diabetes. The peptide treatments (in first clinical trials) are marked by a shift in the cytokine profiles of specific autoimmune T cells from a pro-inflammatory Th-1 response to an anti-inflammatory Th-2 response.
2. Epitopes of HSP's are recognised by the adaptive arm of the immune system (antigen receptors of T cells and B cells)
3. Epitopes of HSP's are targets for regulatory T-cells in both diseases.
   In models of type I diabetes and arthritis, immunisation with HSP has been seen to prevent and to suppress disease. The probable mechanism here is the expansion of microbial (commensal) HSP reactive T cells, tolerised in the gut through mechanisms of mucosal tolerance. This expansion of HSP reactive T cells was possible through both oral and parenteral routes of HSP administration. The expanded T cells are cross-reactive with homologous self-HSP over-expressed in the inflamed (stressed) tissue. And this cross-reactivity of tolerant T cells does lead to regulatory cytokine production at the site of inflammation. For type I diabetes and arthritis first clinical trials in humans have shown the potential of HSP derived peptides to switch cytokine patterns of disease associated T cell specificities into more regulatory cytokine production.

WO95/25744 describes the use of parts of mycobacterial heat shock proteins (HSP65) having mammalian sequence similarity for protection against or treatment of an inflammatory disease, including autoimmune diseases, such as diabetes, arthritic diseases, atherosclerosis, multiple sclerosis, myasthenia gravis, or inflammatory responses due to tumour or transplant rejection. The document is silent about IBD and about how to treat or prevent non-autoimmune diseases such as IBD.

U.S. Pat. No. 6,007,821 describes the use of full length, human heat shock proteins HSP90 and HSP70 for the treatment of autoimmune diseases. Only data for using gp96 (a HSP90 member) to treat insulin dependent diabetes mellitus (IDDM) is provided. The effect is seen only after onset of IDDM and the autoimmune response (i.e. the abnormal immune response to self antigens) is said to be reversed by the treatment with full length human gp96. The heat shock proteins used in the therapeutic treatment in U.S. Pat. No. 6,007,821 are preferably obtained from the patients to be treated, i.e. the patient having developed the autoimmune disease (i.e. they are autologous proteins). Erroneously, ulcerative colitis is mentioned as being an autoimmune disease and, therefore, as being treatable using human, autologous HSP90 or HSP70 proteins or protein complexes.

There still remains a need for methods and compositions suitable for the treatment and/or prevention of non-autoimmune diseases, such as IBD. There also remains a need for methods which can be used to identify or select peptides which, when administered in suitable amounts to humans, treat or prevent one or more IBDs or symptoms thereof.

Dengjel, Schoor et al (2005, PNAS USA 102: 7922-2927) describe experiments which show that fragments of HSP70 family members are loaded into Major Histo-compatibility Complex class II (MHC class II) molecules when human B cells are cultured under conditions of nutrient deprivation and that this occurs primarily for intracellular cytosolic proteins and not for extracellular proteins. This paper is silent about the use of such HSP fragments for induction of disease suppressive immune regulation.

Crotzer and Blum (2005, PNAS USA 102: 7779-7780) describe the molecular and cell biological basis of the HSP70 uploading of MHC class II and review the evidence that cell stress leads to mechanisms of autophagy and that especially HSP70 family member fragments are loaded into MHC class II molecules by the mechanisms of chaperone mediated autophagy (CMA). There is no mentioning of the possible induction of disease suppressive immune regulation.

Paludan et al. (2005, Science 307: 593-595) describe that lysosomal processing after autophagy contributes to MHC class II-restricted surveillance of long-lived endogenous antigens. In the added supplementary material (Supplemental Table 1) it is described that HSC70 and HSP70 are two of the three most frequent cytosolic/nuclear MHC class II natural ligand sources.

Mizushima, Yamamoto et al. (2004, Mol Biol Cell 15: 1101-1111) describe that autophagy is constitutively active and occurs without nutrient deprivation or other cell stress inducing events, in thymic epithelial reticular cells and that such thymic autophagy was more active in newborns. From this one can infer that autophagy contributes to development of T cell repertoire.

Hutszti, Bene et al. (2004, Inflamm Res 53: 551-555) describe experiments aimed at supporting the observation that low levels of antibodies against mycobacterial hsp65 are found in patients with IBD.

Elsaghier et al. (1992, Clin. Exp. Immunology 89: 305-309) describe the measurement of antibody levels to mycobacterial and human heat shock proteins in patients with Crohn's disease, ulcerative colitis and non-tuberculous mycobacterial diseases of the lung. They conclude that the data are not sufficient to imply sensitization with mycobacteria in patients with IBD. Thus, other bacterial proteins may be involved in sensitization.

The origin of inflammatory bowel diseases (IBD) is known to depend on the presence of bacterial gut flora and is regarded as an inappropriate hyper-responsiveness to commensal organisms (Bouma and Strober 2003, Nature Rev. Immunol. 3: 521-533). In surgically excluded ileum of Crohn's patients (no fecal stream) lesions were seen to disappear. Infusion of intestinal contents induces recurrent Crohn's disease (D'Haens et al. 1998, Gastroenterology 114:262-267). Moreover, under germ-free conditions no experimental IBD disease can be induced, unless the gut flora is reconstituted (Chandran et al. 2003, Surgeon 1:125-136, Strober et al. 2002, Annu. Rev. Immunol 20:495-549). Therefore, supposedly, bacterial antigens are the trigger leading to the induction of disease. In IBD such as Crohn's disease no causally related auto-antigens are known to exist, which is in contrast to auto-immune diseases. IBD are, therefore, considered not to be auto-immune diseases such as type 1 diabetes and rheumatoid arthritis are.

Models of IBD have generated evidence for a primary role of anaerobic bacteria (Clostridium, Bacteroides) in the induction of disease (see Verdu et al. 2000, Clin Exp Immunol. 120(1):46-50). Crude sonicates of anaerobic, aerobic gram positive and gram negative bacteria have been administered orally in DSS-induced colitis and only sonicates of anaerobic bacteria were found to reduce severity of experimental colitis (Verdu et al. 2000, Clin Exp Immunol.120(1):46-50).

### SUMMARY OF THE INVENTION

The present inventors have found that HSP70 proteins and/or one or more HSP70-derived peptides can be used for treating and/or preventing one or more Inflammatory Bowel Diseases. The invention, therefore, relates to the peptides, proteins, and mixtures thereof, as such, to compositions comprising one or more of these, as well as to methods for identifying protective HSP70 peptides which are capable of activating HSP70 specific regulatory T cells. Also provided are HSP70-peptide specific T cell lines and hybridomas, which can be used in vitro or in vivo as a source of HSP70-peptide specific regulatory T cells, for research purposes and for use of such specific regulatory T cells in treatment and therapy.

After immunizing mice with full length mycobacterial HSP70 protein and epitope mapping analysis, testing proliferation of T-cell responses in the presence of mycobacterial HSP70-derived peptides (in a screen with 123 overlapping HSP70 peptides, see Wendling et al. 2000, J. Imm. 164: 2711-2717), thirteen mycobacterial 15-mer peptides were identified, which were recognized by mycobacterial HSP70 primed T-cells and were, thus, considered candidates for being involved in the activation of HSP-specific regulatory T-cells. Using a pooling strategy of the 13 peptides (divided into pools based on their homology to mouse homologue HSP70 peptides) in immunizing mice, they found that two of the conserved bacterial peptides (designated ‘B1’ and ‘C1’) induced T cells which were cross-reactive with the mouse homologue HSP70 peptides (‘mB1’ and ‘mC1a’, ‘mC1b’, respectively) i.e. the bacterial peptide-primed T cells cross-reacted with the homologous “self” HSP70 peptides of mouse (i.e. the mouse homologue peptides.

In subsequent experiments administering full lengths mycobacterial HSP70 protein or mycobacterial and/or mouse HSP70 peptides (e.g. a mixture of a mycobacterial peptide having amino acids VLRIVNEPTAAALAY and designated “C1” and mouse homologue HSP70 peptide having amino acid sequence VLRVINEPTAAALAY and designated “mC1a”) prior to inducing Colitis in mice, the oral or intranasal pre-treatment with full lengths HSP70 protein or with the mixture of a bacterial and mammalian homologue HSP70 peptide was able to significantly reduce Colitis progression and symptom severity, indicating that full length HSP70 protein and/or conserved HSP70 peptides can be used to treat and/or prevent Inflammatory Bowel Diseases, such as Colitis. Thus, bacterial HSP70 protein and/or peptides are able to induce proliferation and expansion of specific protective T-cells, which are cross-reactive with “self” HSP70 epitopes (self HSP70 peptides), such as those derived from HSP70 proteins which are induced—locally—by the IBD.

The following peptides and/or mixtures thereof (and composition comprising these or consisting of these) were found to induce a HSP70 specific T-cell response and to be useful in methods for treating and/or preventing IBDs, such as Crohn's disease, granulomatous colitis, ulcerative colitis, lymphocyte colitis, collagenous colitis and Coeliac disease:

1) SEQ ID NO: 1—mycobacterial HSP70 peptide designated “B1” and having the amino acid sequence DEVVAVGAALQAGVL; and variants thereof (comprising e.g. at least 70% sequence identity to this peptide) and longer peptides comprising this peptide or the variants. A variant of SEQ ID NO: 1 is, for example, depicted in SEQ ID NO: 2, which shows the mouse HSP70 homologue peptide designated “mB1” and having the amino acid sequence DEAVAIGAAIQGGVL.

2) SEQ ID NO: 3—mycobaterial HSP70 peptide designated “C1” and having amino acid sequence VLRIVNEPTAAALAY); and variants thereof (comprising e.g. at least 70% sequence identity to this peptide) and longer peptides comprising this peptide or the variants. Variants of SEQ ID NO: 3 are for example depicted in the following two sequences: SEQ ID NO: 4, depicting the mouse HSP70 homologue peptide designated “mC1a” and having amino acid sequence VLRVINEPTAAALAY); and SEQ ID NO: 5 depicting the mouse HSP70 homologue peptide designated “mC1b” and having amino acid sequence VLRIINEPTAAAIAY.

Surprisingly, a peptide having the same amino acid sequence as peptide mC1b identified in the epitope mapping experiment by the inventors (see Examples) had been described in the literature to have been eluted from human MHC class II molecules from a human B cell line kept under stress (nutrient deprivation) conditions to augment autophagy (Dengjel et al. 2005, PNAS102: 7922-7927, see Table 1, human peptide VLRIINEPTAAAIAY) and overlapping peptides had been described to be present in human MH class II by others (see Examples). The inventors hypothesized, without being bound by any theory, that therefore also other HSP70-derived peptides eluted from MHC class II molecules (and/or their bacterial homologues) are suitable for treating and/or preventing one or more IBD, such as Crohn's disease, granulomatous colitis, ulcerative colitis, lymphocyte colitis, collagenous colitis and/or Coeliac disease. Thus, a further group of peptides is provided which are useful according to the invention:

3) HSP70 protein derived peptides, which bind the MHC class II on cells of the subject to be treated and which induce T cells that are cross-reactive with the homologous “self” HSP70 peptides of the subject.

Screening the literature for HSP70-derived peptides which are presented on (bind) MHC class II molecules (i.e. accommodated in the MHC class II cleft and which can be eluted therefrom), about 30 peptides were identified (see Examples and SEQ ID NO: 17-47), all of which are encompassed herein as being suitable for the treatment or prevention of one or more IBDs. Thus, in one embodiment of the invention compositions comprising one or more HSP70-derived peptides (and compositions comprising or consisting of these), which are capable of binding mammalian, especially human, MHC class II molecules, are provided herein for the activation of HSP70 specific regulatory T cells and, thus, for the treatment and/or prevention of one or several IBDs.

Also, a method of using one or more MHC class II—eluted peptides of HSP70 family members for (preparing a medicament for) the treatment or prevention of one or more IBDs is provided. Further, a method of using one or more longer HSP70 peptides, comprising these eluted peptides, is provided herein. In another embodiment a method of using a full length HSP70 protein for (preparing a medicament for) the treatment or prevention of one or more inflammatory bowel diseases (IBD) is provided herein. Similarly, pharmaceutical, nutritional or food supplement compositions comprising one or more HSP70 peptides and/or full lengths HSP70 proteins are provided, which can be used for immunizing or pre-immunizing mammals, especially humans, against IBDs, and/or for treatment of IBDs after onset of symptoms.

In a further embodiment a method for identifying HSP70-derived peptides suitable for the treatment and/or prevention of one or more IBDs is provided, comprising:
a) Providing one or more HSP70-derived peptides, preferably from a bacterial or mammalian HSP70 protein; and
b) optionally determining whether a HSP70-derived peptide is capable of binding MHC class II molecules, i.e. binding the MHC class II cleft, or being elutable from the MHC class II molecule;
c) optionally testing the capacity of the peptide to induce peptide specific T-cell which are cross-reactive with the homologous self-peptide of a mammal (especially a human or animal model or cell lines) in a cross-reactivity assay and selecting those peptides which do show cross-reactivity; and
d) administering a composition comprising or consisting of one or more of the peptides in an animal model of an IBD disease to determine the in vivo protective activity, and comparing disease development and/or symptoms between treated and control animals, thereby verifying that the one or more peptides are suitable for treatment and/or prevention of one or more IBDs, and
e) using the one or more peptides for the preparation of a medicament for the treatment and/or prevention of IBDs in humans or animals and in a method for treating and/or preventing IBDs in subjects, especially humans or domesticated animals, such as farm animals and companion animals.
   Thus, in one embodiment the peptide is derived from a HSP70 protein and comprises a T cell epitope that protects against induced IBD in model animals such as mice (see step d). In another embodiment the peptide comprises or consists of part of the full HSP70 protein, or a panel of peptides of the full HSP70 protein, or a mixture of two or more HSP70 derived peptides and protects against experimental IBD when administered to mice (see step d).

Also a method for the treatment or prevention of inflammatory bowel diseases (IBD) in a human or animal subject is provided, comprising administering to a subject in need thereof a therapeutically or prophylactically effective amount of one or more HSP70 peptides (or variants thereof, or longer peptides comprising the shorter peptides) and/or one or more full length HSP70 proteins (see step e).

### GENERAL DEFINITIONS

“IBD” refers herein to Inflammatory Bowel Diseases, a chronic inflammation of the gastrointestinal tract, comprising or consisting of the following diseases: Crohn's disease, granulomatous colitis, ulcerative colitis, lymphocyte colitis, collagenous colitis and Coeliac disease.

“Autoimmune diseases” refers to diseases such as insulin-dependent diabetes mellitus (type 1 diabetes), artherosclerosis, myasthenia gravis, experimental autoimmune encephalomyelitis, etc. wherein the primary disease initiating and maintaining immune response is directed against auto-antigens (self antigens; i.e. antigens of normal cellular components) of the subject. Autoantibodies and T cells are produced, which are specific for such autoantigens.

“Non autoimmune diseases” refers herein to diseases wherein the primary disease initiating and maintaining immune response is not directed against auto-antigens (self antigens) of the subject, but against non-self antigens (foreign antigens). For example, IBDs are non autoimmune diseases. In this/the latter case autoimmune responses are not responsible for initiating and/or maintaining the inflammation. In the case of Crohn's disease the non-autoimmune nature of the disease was demonstrated in surgically constructed blind-loops, where removal of fecal content led to resolution of the disease, whereas re-infusion of fecal contents led to disease recurrences (Rutgeerts et al. 1991, Lancet 338:771-774).

“Subject(s)” are herein mammals, especially humans and/or domesticated animals, especially farm animals (cows, horses, pigs, etc.) or companion animals (dogs, cats, rabbits, etc.). The term ‘model animal’ usually refers herein to non-human animals, especially non-human mammals such as mice, rats, rabbits, etc.

The term “antigen” (or immunogen) includes reference to a substance capable of eliciting an adaptive immune response, i.e. to induce production of antigen recognition molecules (especially antigen-specific or cross-reactive T cells) to which the antigen is specifically immunoreactive. The specific immunoreactive sites within the antigen are known as “epitopes” (or antigenic determinants). Herein proteins or protein fragments peptides) consisting of, or comprising, one or more epitopes of bacterial and/or mammalian HSP proteins capable of preventing and/or treating one or more IBDs are provided. These epitopes are also referred to as “protective epitopes”.

“T cell epitope” refers to the epitopes recognized by the T cell receptors. Upon binding of the epitope, an immune response is mounted in the subject.

“Enteral” refers herein to the delivery directly into the gastrointestinal tract of a subject (e.g. orally or via a tube, catheter or stoma). “Nasal” refers to administration via the nose.

“Percentage” or “average” generally refers to percentages of averages by weight, unless otherwise specified or unless it is clear that another basis is meant.

“Sequence identity” and “sequence similarity” can be determined by alignment of two amino acid sequences or two nucleotide sequences using global or local alignment algorithms. Sequences may then be referred to as “substantially identical” or “essentially similar” or as “variants” when they (when optimally aligned by for example the programs GAP or BESTFIT using default parameters) share at least a certain minimal percentage of sequence identity (as defined below). GAP uses the Needleman and Wunsch global alignment algorithm to align two sequences over their entire length, maximizing the number of matches and minimises the number of gaps. It is most suitable for aligning sequences of similar lengths. Generally, the GAP default parameters are used, with a gap creation penalty being higher than the extension penalty (e.g. gap opening penalty=10 and gap extension penalty=0.5). For nucleotides the default scoring matrix used is nwsgapdna and for proteins the default scoring matrix is Blosum62 (Henikoff & Henikoff, 1992, PNAS 89, 915-919). Sequence alignments and scores for percentage sequence identity may be determined using computer programs, such as the GCG Wisconsin Package, Version 10.3, available from Accelrys Inc., 9685 Scranton Road, San Diego, Calif. 92121-3752 USA. Also, EmbossWin version 2.10.0 can be used, using the program ‘needle’ (which corresponds to GAP) with the same parameters as for GAP above. For sequences of different lengths preferably local alignment algorithms such as the Smith Waterman algorithm are used, such as provided by the program ‘water’ of e.g. EmbossWin version 2.10.0, using default parameters (gap opening penalty of 10.0 and a gap extension penalty of 0.5) or programs such as BLAST or FASTA.

The terms “protein” or “polypeptide” are used interchangeably and refer to molecules consisting of a chain of amino acids, without reference to a specific mode of action, size, 3 dimensional structure or origin. A “fragment” or “portion” of a protein, or a peptide comprising a fragment of a natural protein, may thus still be referred to as a “protein” or “peptide”. An “isolated protein” is used to refer to a protein which is no longer in its natural environment, for example in vitro or in a recombinant bacterial host cell.

Depending on the context, the term “homologue” or “homologous” refers to sequences which are descendent from a common ancestral sequence. If desired, the term can be specified by referring to orthologs and paralogs, see http://www.ncbi.nlm.nih.gov/Education/BLASTinfo/Orthology.html). Orthologs generally retain the same function in a different species. Paralogs, in contrast evolved different (possibly related) functions. Bacterial heat shock proteins and mammalian heat shock proteins of the HSP70 family are, thus, herein referred to as homologues, as are HSP70 proteins of different species of bacteria or of different species of mammals (e.g. mouse and human HSP70 proteins). A “mammalian homologue” of a bacterial HSP70-derived peptide, will therefore be at least 70%, preferably at least 80%, 90% or more identical in amino acid sequence to the bacterial peptide, when aligned pairwise using e.g. a local alignment algorithm (e.g. Smith Waterman).

The terms “homologous” and “heterologous” may also be used to refer to the relationship between a nucleic acid or amino acid sequence and its host cell or organism, especially in the context of transgenic cells/organisms. A homologous sequence is thus naturally found in the host species, while a heterologous sequence is not naturally found in the host cell.

“Stringent hybridisation conditions” can be used to identify nucleotide sequences, which are substantially identical to a given nucleotide sequence. Stringent conditions are sequence dependent and will be different in different circumstances. Generally, stringent conditions are selected to be about 5° C. lower than the thermal melting point (Tm) for the specific sequences at a defined ionic strength and pH. The Tm is the temperature (under defined ionic strength and pH) at which 50% of the target sequence hybridises to a perfectly matched probe. Typically stringent conditions will be chosen in which the salt concentration is about 0.02 molar at pH 7 and the temperature is at least 60° C. Lowering the salt concentration and/or increasing the temperature increases stringency. Stringent conditions for RNA-DNA hybridisations (Northern blots using a probe of e.g. 100 nt) are for example those which include at least one wash in 0.2×SSC at 63° C. for 20 min, or equivalent conditions. Stringent conditions for DNA-DNA hybridisation (Southern blots using a probe of e.g. 100 nt) are for example those which include at least one wash (usually 2) in 0.2×SSC at a temperature of at least 50° C., usually about 55° C., for 20 min, or equivalent conditions. See also Sambrook et al. (1989) and Sambrook and Russell (2001).

In this document and in its claims, the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the element is present, unless the context clearly requires that there be one and only one of the elements. The indefinite article “a” or “an” thus usually means “at least one”, e.g. “a cell” refers also to several cells in the form of cell cultures, tissues, whole organism, etc. It is further understood that, when referring to “sequences” herein, generally the actual physical molecules with a certain sequence of subunits (e.g. amino acids) are referred to.

### DETAILED DESCRIPTION

The invention pertains to the use of one or more heat shock protein 70 (HSP70) derived peptides eluted from and/or capable of binding to MHC class II molecules, and/or to one or more full length HSP70 proteins, for the treatment and/or prevention of IBD, especially various forms of Colitis, such as those selected from the group consisting of Crohn's disease, Granulomatous Colitis, Ulcerative Colitis, Lymphocyte Colitis, Collagenous Colitis and Coeliac disease.

### Peptides and Proteins for Use According to the Invention

Heat shock proteins are universal proteins, which carry out important house-keeping functions of prokaryotic and eukaryotic cells. They play an important role as chaperones in protein folding and in rescuing the cell from stress conditions. They are classified into different families on the basis of their monomeric molecular weight. Thus proteins of the family HSP70 have a molecular weight of about 70 kDa. The main families are HSP 10, 40, 60, 70, 90 and 100. Many mammalian HSP family members have highly conserved microbial homologues.

The present inventors found that peptide fragments of mycobacterial HSP70 and of mammalian HSP70 proteins, as well as full lengths bacterial and/or mammalian HSP70 proteins can be used to treat and/or prevent IBD in mammals, especially in humans. They then found that the human homologue of one of the identified mouse peptides (designated mC1b) had been described in the paper of Dengjel et al. (PNAS 2005, supra) as being presented on MHC class II molecules. In the paper it is shown and argued that cytosolic proteins, especially under conditions of cell stress, are routed into the MHC class II pathway of antigen presentation through autophagy. In autophagy three distinct mechanisms are operative of which chaperone mediated autophagy (CMA) is one mechanism. In CMA HSC70 is a transporter molecule which assists Lamp-2 in its function to transfer cytosolic proteins into the lysosome for MHC class II presentation. Due to this mechanism of CMA, HSC70 (HSP70 family of proteins) fragments are preferentially intersecting with the MHC class II loading compartment and are captured in the MHC class II binding cleft. In the Dengjel et al. paper HSP70 peptides are found to dominate the cytosolic peptide repertoire that is retrieved from MHC class II and are upregulated under conditions that augment autophagy (nutrient starvation). Nowhere in the paper there is any interpretation of what this could mean for HSP specific CD4+ T cell recognition, but having found that similar peptides (designated herein mC1a and C1) are suitable for treatment and/or prevention of IBDs, the inventors concluded that HSP70-derived peptides which are capable of being bound to MHC class II molecules and which induce CD4+ T-cells that are cross-reactive to the homologous “self” HSP70 peptide can be used to treat and/or prevent IBD in mammals, especially humans. The term “treatment” refers herein to any reduction or alleviation of disease symptoms and/or progression, after administration of a suitable amount of one or more HSP70-derived peptides and/or HSP70 proteins, whereby the administration takes place one or more times after the IBD has been diagnosed. This term encompasses thus a reduction in disease symptoms and/or progression in the treated subject (relative to the untreated subject), as well as a complete healing of the subject. The term “prevention” refers to the prophylactic administration of compounds or compositions according to the invention to subjects which have not yet been diagnosed with one or more IBDs, but which may, for example, be at risk of developing disease.

The inventors were also guided by the paper of Mizushima et al. (Molecular Biology of the Cell, 2004, Vol. 15: 1101-1111) where it is shown that autophagy takes place in the thymus, especially in newborns (ref to be added), in the thymic epithelium cells. The inventors reasoned that thymic epithelium cells are involved with positive selection of the T cell repertoire. This would open the possibility that HSP70 becomes uploaded in MHC class II at the location where, and at the time when, positive selection of the T cell repertoire is organised. In other words, from this the inventors inferred that thymic selected HSP70 derived peptides would possibly lead to the formation of a T cell repertoire of regulatory T cells with specificity for HSP70 peptides.

The present inventors have combined these pieces of information with their own experimental data (see Examples) in concluding that HSP70 family members derived peptides in the binding cleft of MHC class II molecules of cell lines subjected to conditions of raised autophagy, such as stress and nutrient deprivation, have the quality of inducing regulatory T cell responses and are suitable for the treatment and/or prevention of IBDs.

The inventors have tested the mycobacterial HSP70 derived peptide designated C1 (SEQ ID NO: 3), in a mixture with the cross-reactive mammalian (mouse) homologue thereof (the HSP70 homologue peptide designated mC1a, SEQ ID NO: 4), for its capacity to induce regulatory T cell responses (to the homologous mouse HSP70 peptide) in the model of TNBS induced colitis in Balb/c mice. The experiment showed that nasal administration of this peptide (peptide mixture) suppressed TNBS induced colitis disease when administered after TNBS skin sensitization and prior to induction of disease by rectal TNBS administration (see Examples).

Inventors have also tested the complete mycobacterial HSP70 molecule in models of experimental IBD. In this case the mycobacterial HSP70 of SEQ ID NO: 55 was tested. For this purpose both the models of TNBS induced IBD and the model of DSS induced IBD were used. In both models the full lengths mycobacterial HSP70 protein, when administered orally and/or nasally, inhibited disease development.

Thus, the present inventors found that nasal and/or oral administration of HSP70 derived peptides and/or complete HSP70 protein in the rodent TNBS and DSS models of colitis is highly disease suppressive (see Examples). Both a prophylactic effect as well as a treatment effect was observed.

Thus, in one embodiment a protective epitope, or protein or protein fragment (peptide) consisting of or comprising protective epitopes are provided, as are compositions comprising one or more of these. Especially in one embodiment one or more HSP70 peptides, or longer polypeptides comprising such a peptide, are provided for the preparation of a compositions (e.g. a medicament or vaccine) for the treatment and/or prophylaxis of IBDs. Also a panel of HSP70 peptides is provided, whereby a panel includes overlapping peptides, which together cover the full length HSP70 protein. Part or all of such a panel may also be used to prepare compositions according to the invention.

The term “HSP70 protein” refers herein to full length proteins such as full length HSP70 proteins of human, animal (e.g. mammalian), bacterial or other origin and having been classified as belonging to the HSP70 family of proteins. The term includes both mature proteins (lacking N-terminal signal peptides) and non-processed proteins (comprising the N-terminal signal peptide). It also includes “variants” such as proteins having one or more amino acids deleted, replaced or inserted relative to the native protein. Variants may comprise at least 50%, 60%, 70%, 80%, 85%, 90%, 95%, 98% or 99% or more amino acid identity to the native protein (i.e. the HSP70 protein found in nature in the organism), when aligned over the entire length, using preferably a global alignment algorithm. Examples of HSP70 proteins include the Mycobacterial HSP70 protein depicted in SEQ ID NO: 55 and variants thereof, the human HSP70 proteins depicted in SEQ ID NOs: 48-54 and variants thereof, such as proteins comprising at least the above % amino acid sequence identity to SEQ ID NOs: 55 and/or 48-54.

The term “HSP70 derived peptide” or “HSP70 peptide” or “HSP70 fragment” refers herein to fragments of the above described HSP70 proteins and/or protein variants, such as fragments comprising or consisting of at least 5, 6, 7, 8, 9, 10, 11, 12, 13, 14, 15, 16, 17, 18, 19, 20, 25, 30, 40, 50, 60, 70 or more consecutive amino acids of a HSP70 protein or variant as defined above. Thus, when aligned with a full length HSP70 protein or variant (as defined above) using a local alignment algorithm the at least 5, 6, 7, 8, 9, 10, 11, etc. consecutive amino acids of the peptide will match the full lengths protein or variant by 100%. A HSP70 derived peptide may further comprise additional amino acids e.g. at one or both ends. These further peptides need not necessarily be complementary to the natural HSP70 amino acid sequence. The variability in length reflects the fact that different lengths variants can bind the MHC class II molecules. Non-limiting examples of HSP70 derived peptides according to the invention consist of, or comprise, SEQ ID NO: 1-5, or variants thereof, such as peptides which comprise 1, 2, 3, 4 or 5 amino acid insertions or additions, replacements or deletions with respect to SEQ ID NO: 1-5 and which are still protective in vivo. Other preferred peptides are peptides consisting or, or comprising, SEQ ID NO: 17-47, or variants thereof, such as peptides which comprise 1, 2, 3, 4 or 5 amino acid insertions or additions, replacements or deletions with respect to SEQ ID NO: 17-47 and which are still protective in vivo. Obviously, the HSP70 proteins and peptides according to the invention are functional in vivo in treating and/or preventing IBDs, and functionality can be tested or determined as explained further below.

A “panel” of HSP70 peptides refers to a collection of at least two, three, four, five, and so forth, up to at least 50, 100, 120, 130, 150, 200, 300 or more peptides which overlap by one or more amino acids (preferably at least by 3, 5, 8, 9, 10, 11 amino acids or more), so that the entire HSP70 protein is covered by the collection, i.e. alignment of the overlapping regions could reconstitute the entire HSP70 protein or protein variant. For example, a panel of 15-mer peptides which overlap by 10 amino acids results in a panel of 123 peptides. The panel of peptides will be smaller or larger with different peptide length and/or overlap.

In principle, any HSP70 protein, or fragment thereof, may be used, such as any HSP70 protein of a microbial (e.g. bacterial, such as from Mycobacterium, etc.) or a mammalian (e.g. rat, mouse, human etc.) origin. HSP70 proteins of a wide variety of organisms have been cloned and sequenced and can, proteins and/or peptides thus be produced by e.g. recombinant DNA techniques, synthesized chemically or be purified from natural sources using methods well known in the art. When using full lengths HSP70 proteins (or a panel of peptides, e.g. covering the full length protein), it is one embodiment that the full length protein is not of human origin, especially when it is used for the treatment and/or prevention of ulcerative colitis. This embodiment, thus, comprises the use of full-length non-human HSP70 protein for the treatment or prevention of ulcerative colitis. The non-human HSP70 protein (or variant thereof) may originate from microbial (e.g. bacterial) and/or non-human animals, i.e. it may be naturally found in other mammals than Homo sapiens. HSP70-derived peptides may be fragments of HSP70 proteins of any origin, e.g. bacterial, synthetic, artificial, mammalian (rat, mouse, bovine, etc.) or human.

Preferably the HSP70 proteins and/or HSP70 peptides comprise or consist of epitopes which are functional in vivo, i.e. they prevent or treat one or more IBDs when administered in suitable concentrations. Such peptides are preferably capable of a) binding the MHC class II molecules of a subject (e.g. human), and, optionally (b) they are capable of activating regulatory T-cells (CD4+ cells) which are cross-reactive with the homologous “self” HSP70-peptide of the subject (e.g. human or animal) or of a mammalian model animal, such as mouse and (c) when administered to an animal or human subject the peptide(s) is/are able to treat and/or prevent one or more IBDs. The above capabilities of the peptides can be tested as described in detail elsewhere herein, but basically capability a) can be determined by either identifying peptides from the scientific literature, which have been described to have this capability and/or by MHC class II binding assays in vitro or in vivo; capability b) can be tested by in vitro priming and capability c) can be tested by using e.g. animal models and/or in human trials. In animal models also humanised MHC transgenic animals can be used.

Thus, in one embodiment a peptide having a specific amino acid sequence is used as such, e.g. the composition comprises or consists of a plurality of molecules of one of SEQ ID NO: 1-5, or 17-47, or other individual HSP70 derived peptides.

In another embodiment a mixture of two or more different HSP70 derived peptides is provided. These different peptides may be from the same HSP70 protein (e.g. derived from a bacterial HSP70, e.g. from Mycobacterial HSP70, such as SEQ ID NO: 55) or from different HSP70 proteins, such as from homologues of different species, e.g. one peptide being from mouse or human HSP70 and one peptide being from bacterial HSP70. Also mixtures of one or more HSP70 peptides and one or more full length HSP70 proteins may be used. In a preferred embodiment a composition for the treatment and/or prevention of IBDs comprises or consists of at least one microbial and at least one a mammalian HSP70 peptide, whereby the bacterial and the mammalian peptide are preferably homologues of one another, i.e. they are very similar in their amino acid sequence and cross-reactive. For example, a preferred composition comprises peptides of SEQ ID NO: 3 (bacterial ‘C1’) and one or more of the mouse homologues (SEQ ID NO: 4 and/or 5, or other variants thereof).

When referring to “a heat shock protein” it is understood that the protein occurs in bacteria or mammalian cells in nature, but may be produced or isolated by various means. For example, it may be produced by recombinant DNA technology, whereby the nucleotide sequence encoding the protein (or protein fragment) is used to transform or transfect a host cell, which then produces the protein or protein fragment. Nucleic acid sequences (cDNA, RNA and genomic DNA) encoding HSPs are available in the art or may be made by chemical synthesis, and the methods for recombinant production of the protein or protein fragment (peptide) are routine. Similarly, nucleic acid hybridization techniques (for example using stringent hybridization conditions) may be used to isolate genes encoding HSPs. Alternatively, the protein or protein fragment may be purified or partially purified from natural sources (e.g. mammalian cells, bacteria or plants) or may be synthesized chemically. For example, the peptides can be synthesised by the well-known Merrifield solid-phase synthesis method in which amino acids are sequentially added to a growing chain. See Merrifield (1963), J. Am. Chem. Soc. 85:2149-2156; and Atherton et al., “Solid Phase Peptide Synthesis,” IRL Press, London, (1989). Automatic peptide synthesisers are commercially available from numerous suppliers, such as Applied Biosystems, Foster City, Calif.

In the broadest sense, any HSP70 protein which is known to harbour peptide sequences that have the capacity to bind MHC class II molecules of a subject is provided. This capacity can be tested using various methods known in the art. For example, a complete overlapping set of e.g. 15-mer peptides covering the whole HSP70 protein can be tested in MHC class II binding assays, see Peptide electrophoretic mobility shift assay, Mittelman A, Lucchese G, Stufano A, Kanduc D., Degenerate binding of tyrosinase peptides to MHC II Ad/Ed molecules. J Exp Ther Oncol. (2007) 6:231-9. See also competition based binding assay, described by Joosten, I., M. H. M. Wauben, M. C. Holewijn, K. Reske, L. O. Pedersen, C. F. Roosenboom, E. J. Hensen, E. van Eden, S. Buus. (1994). Direct binding of autoimmune disease related T cell epitopes to purified Lewis rat MHC class II molecules. Int. Immunol. 6:751. See also United States patent application number US2004224009. Preferably, such methods are used to test whether one or more peptides are capable of binding to MHC class II molecules.

“Fragments” refer herein to peptides comprising at least 6 or 7, more preferably at least 8 or 9, more preferably 10, 11, 12, 13, 14, 15, 16, 17, 18, 20, 25, 30, 40, 50, 55, 60, 70 or more consecutive (contiguous) amino acids of any of the HSP70 according to the invention. As already mentioned, this is relevant especially because MHC class II binding peptides have varying lengths and multiple length variants can bind similar MHC class II molecules. In one embodiment a fragment is not longer than 40, 30, preferably 25, 20 or 15 amino acids.

Both full length HSP70 proteins and fragments according to the invention are preferably “functional”, i.e. they consist of or comprise one or more peptide sequences that have the capacity to treat and/or prevent one or more IBDs when administered in vivo. Thus, ultimately, in vivo animal models of IBDs should be used to test functionality as described in the Examples and as known in the art.

The peptides preferably are able to bind NMC class II molecules of a subject (see above). Although not all HSP70-derived peptides, which are able to bind MHC class II molecules, will be able to induce protective T cell responses, a pre-selection of potentially functional HSP70-derived peptides (T cell epitopes) can be made by selecting HSP70 sequences that are found in the clefts of MHC class II molecules, knowing that roughly 50% of MHC bound sequences will be utilised as T cell epitopes by the immune repertoire. Following this pre-selection based on MHC class II binding capacity, the functionality can then be further tested by testing the ability of the peptide(s) to induce T-cells (like with an in vitro human T-cell sensitization assay, e.g. see: page 3239 RH Column “Cellular Assay” and page 3243 LH column in Halder T, Pawelec G, Kirkin A F, Zeuthen J, Meyer H E, Kun L, Kalbacher H., Isolation of novel HLA-DR restricted potential tumor-associated antigens from the melanoma cell line FM3., Cancer Res., 1997, 57:3238-44) and which are cross-reactive with homologous “self” peptides by analyzing them in the in vitro sensitization assay, and/or by testing the in vivo functionality in an animal model, whereby test animals are administered with potentially protective peptides and those peptides which significantly reduce or prevent IBD (compared to suitable controls) in the animals are selected.

To test cross-reactivity, i.e. the capacity of selected peptides to induce T cells cross-reactive with homologous self-proteins, the following method can be used (see also Examples):

T cells activated in vitro with the defined (e.g. microbial) HSP70 epitopes, can then be restimulated with the homologous self peptide (in the mouse model the mouse HSP, either as a recombinant protein or purified from stressed cells or tissue or as elevated levels of MHC-peptide complexes on stressed antigen presenting cells) or the homologous peptide. Any sign of activation (see above) can be taken as an indication of cross-reactivity of the test (e.g. microbial) epitope with the self protein/peptide. Initial testing can be carried out with a synthetic peptide based on the sequence of the self protein/peptide, but final proof for cross-reactivity with the protein/peptide itself, either in isolated form or expressed on cells, is preferably obtained in T cell activation tests with (stressed) antigen presenting cells (over)expressing and presenting the self HSP-peptide, in order to exclude cryptic epitopes.

An easy way of determining the most suitable fragment is to generate overlapping peptides (for example overlapping pentamers, hexamers, heptamers, or decamers; i.e. short consecutive amino acids) of a full length bacterial HSP protein or mammalian/human HSP protein and to screen these overlapping peptides for their protective effect. For example, by administration in a rodent model of Colitis, as described in the Examples.

In one embodiment a complete HSP70 protein is used, as defined above. For example the Mycobacterial HSP70 (SEQ ID NO: 55) consists of 625 amino acids in length. Fragments thereof, or of a variant thereof can be used. In another preferred embodiment a fragment comprising or consisting of at least about 6, 7, 8 or more consecutive amino acids of SEQ ID NO: 55, or a variant thereof, is used, for example SEQ ID NO: 1-5, or variants thereof.

In yet another embodiment according to the invention, HSP proteins for use according to the invention comprise all proteins having at least 35%, 40%, 45%, 50%, 60%, 70%, 80%, 90%, 95%, 98%, 99% or more (100%) amino acid sequence identity (over the full length of the protein) to any one of SEQ ID NO: 55 or 48-54 (also referred to as “variants” of SEQ ID NO: 55, or 48-54), or one or more (functional) fragments thereof (as defined above), irrespective of the origin of the protein and irrespective of whether it occurs naturally (in nature).

In these embodiments, it is not required that the protein occurs naturally in bacteria or mammals (e.g. humans), as herein variants of the naturally occurring HSP protein are used. Such variants may, of course, also occur naturally in bacteria or mammals. These variants (and fragments thereof) may be generated by methods known in the art, such as site directed mutagenesis, de novo chemical synthesis, recombinant expression of nucleic acid sequences comprising deletions, replacements, or additions of one or more nucleotides, gene shuffling techniques, etc. For example small modifications to a DNA sequence such as described above can be routinely made, i.e., by PCR-mediated mutagenesis (Ho et al., 1989, Gene 77, 51-59., White et al., 1989, Trends in Genet. 5, 185-189). More profound modifications to a DNA sequence can be routinely done by de novo DNA synthesis of a desired coding region using available techniques.

Preferred fragments of the bacterial HSP70 (as depicted in SEQ ID NO: 55) consist of or comprise the following amino acids:
amino acid 141-155 of SEQ ID NO: 55 (which is also depicted as SEQ ID NO: 3) and variants thereof;
amino acids 342-356 of SEQ ID NO: 55 (also depicted in SEQ ID NO: 1) and variants thereof;
bacterial homologues of SEQ ID NO: 17-47 (mammalian HSP70 fragments), or variants thereof;
amino acids or any other fragment of at least 6, 7, 8, 9, 10, 11, 12, 13, 14, 15, 16, 17, 18, 20, 30, 40, 50 or more, consecutive amino acids of SEQ ID NO: 55 or variants thereof. Most preferred are fragments which are highly conserved among HSP70 proteins.

Preferred fragments of mammalian homologue HSP70 proteins from e.g. mouse, rat, rabbit, bovine, human, etc. sources (e.g. as depicted in SEQ ID NO: 48-54 for human HSP70 proteins) consist of, or comprise the following amino acids:
mammalian homologues of SEQ ID NO: 1, such as for example SEQ ID NO: 2 (mouse homologues); or amino acids 366-380 of SEQ ID NO: 48 (human homologue hspa1a), or amino acids 368-382 of SEQ ID NO: 49 (human homologue hspa11); amino acids 369-383 of SEQ ID NO: 50 (human homologue hspa2); amino acids 391-405 of SEQ ID NO: 51 (human homologue hspa5); amino acids 368-382 of SEQ ID NO: 52 (human homologue hspa6); amino acids 366-380 of SEQ ID NO: 53 (human homologue hspa8); amino acids 414-428 of SEQ ID NO: 54 (human homologue hspa9), or homologs of other mammalian HSP70 proteins or variants, such as sequences comprising at least 70% amino acid identity to SEQ ID NO: 1, or more (as defined above).
mammalian homologues of SEQ ID NO: 3, such as for example SEQ ID NO: 4 or 5 (mouse homologues); or amino acids 168-183 of SEQ ID NO: 48 (human homologue hspa1a), or amino acids 171-185 of SEQ ID NO: 49 (human homologue hspa11); amino acids 170-184 of SEQ ID NO: 50 (human homologue hspa2); amino acids 195-209 of SEQ ID NO: 51 (human homologue hspa5); amino acids 171-185 of SEQ ID NO: 52 (human homologue hspa6); amino acids 169-183 of SEQ ID NO: 53 (human homologue hspa8); amino acids 216-230 of SEQ ID NO: 54 (human homologue hspa9), or homologues of other mammalian HSP70 proteins or variants, such as sequences comprising at least 70% amino acid identity to SEQ ID NO: 3, or more (as defined above).
amino acids or any other fragment of at least 6, 7, 8, 9, 10, 11, 12, 13, 14, 15, 16, 17, 18, 20, 30, 40, 50 or more, consecutive amino acids of any one of SEQ ID NO: 48-54 or variants thereof (such as homologues from other mammals). Most preferred are fragments which are highly conserved among HSP70 proteins.
SEQ ID NO: 17-47, or variants thereof, such as homologues thereof from other mammalian species or other mammalian HSP70 proteins.

In one embodiment it is preferred that only one protein or protein fragment is used, while in another embodiment mixtures of proteins and/or fragments of different amino acid sequence may be used. Thus, for example the whole HSP70 protein may be mixed/combined with one or more fragments of a HSP70 protein. Alternatively, proteins and/or fragments of different HSP70 proteins may be mixed. For example, a whole HSP70 protein from a bacterium may be mixed/combined or co-administered with one or more specific mammalian HSP70-derived peptides. Thus, also bacterial and mammalian (e.g. human) HSP70 proteins, or functional fragments thereof, may be mixed. Equally mixtures of HSP70 proteins or fragments, of the same or different HSP70 protein, may be mixed. Mixtures comprise both single compositions, which comprise the two or more proteins and/or peptides or, alternatively, separate compositions (referred to as kits) which are co-administered (together, or one shortly after the other), so that only after administration the two or more proteins and/or fragments are combined in vivo, in one subject.

The mixture or kit may also comprise a panel of HSP70 peptides, or part of such a panel. For example, at least 3, 4, 5, or more overlapping peptides of each e.g. 10, 12, 15, 16 or more amino acids in length, which together could reconstitute at least part or even the full HSP70 protein, can be used together in one composition or co-administered so that they are combined in vivo. In case only part of the HSP70 protein is encompassed by the panel, it is preferred that the overlapping peptides make up at least 10, 20 or 25%, preferably at least 30, 40 or 50% of the full HSP70 protein. Also, preferably the peptides preferably overlap to form one or two contigs only. Thus, they may be from two regions of the full lengths protein, e.g. from the region including amino acid 150 of SEQ ID NO: 55 and including amino acid 350 of SEQ ID NO: 55. The peptides preferably overlap by at least one amino acid, more preferably by at least 2, 3, 4, or 5 amino acids.

In yet another embodiment of the invention protective epitopes, or proteins or protein fragments consisting of or comprising protective epitopes are provided, whereby mammalian, such as mouse or human HSP70 proteins, or variants thereof, or fragments of any of these, are used to prepare compositions (or medicaments) for the treatment and/or prophylaxis of one or more IBDs in humans or animals, such as farm animals or companion animals. “Human heat shock 70 proteins” refer to proteins occurring naturally in Homo sapiens, such as those depicted in SEQ ID NO 48-54. Non-human, mammalian heat shock 70 proteins are variants of the human ones, comprising at least 50%, 60%, 70%, 80%, 90%, 95%, 98% or more identity to the human proteins (see definition elsewhere herein). Throughout the description, the embodiments described for bacterial HSP70 proteins and fragments apply equally to mammalian, especially mouse and human HSP70, as well as variants and fragments thereof, and vice versa. Thus, in one embodiment thus also full length human HSP70 protein (or variants or a panel of peptides) may be used to treat human (or animal) subjects, especially to treat or prevent one or more IBDs selected from Crohn's disease, granulomatous colitis, ulcerative colitis, lymphocyte colitis, collagenous colitis and Coeliac disease.

In another embodiment the full length human HSP70 is preferably not used in human subjects for treating or preventing the IBD ulcerative colitis, as herefore full length non-human HSP70 proteins are preferred.

In a different embodiment, preferably full length, non-human HSP70 (or variants or a panel of peptides) may be used to treat human or animal subjects, such as non-human mammalian HSP70 protein (e.g. mouse, rat, bovine, etc).

In another embodiment only bacterial HSP70 proteins and/or peptides are used, i.e. no mammalian peptides are used in the methods and compositions.

In a further embodiment the methods and compositions relate to the use of treatment or prevention of one or more IBDs, wherein the IBDs are non-autoimmune diseases; in one embodiment the IBD is not ulcerative colitis.

Human or mammalian heat shock proteins according to the invention comprise all human or mammalian HSP proteins having at least 35%, 40%, 45%, 50%, 60%, 70%, 80%, 90%, 95%, 98%, 99% or more amino acid sequence identity (over the full length of the protein) to a HSP70 protein found in humans or other mammals, for example to the amino acid sequence depicted in SEQ ID NO: 48-54. Also encompassed are fragments of any of these, especially functional fragments. “Fragments” refer herein to peptides comprising at least 6, 7 or 8, more preferably 9, 10, 11, 12, 13, 14, 15, 16, 17, 18, 19, 20, 30, 40, 50, 60, 70, or more consecutive (contiguous) amino acids of any of the human or mammalian HSP70s or variant according to the invention. Again, both the whole protein or the fragments used are preferably “functional”, i.e. they consist of or comprise one or more protective epitopes and are capable of inducing the production of cross-reactive T-cells when administered to a subject and, especially, to reduce or prevent IBD or one or more symptoms associated with IBD. Thus, to test functionality of a full length protein or fragment, the activation of cross-reactive T cells may be measured as described above or as in the Examples and/or the in vivo functionality may be assessed in an animal model of IBD.

It is understood that the invention also concerns peptide analogues, or proteins or proteins fragment comprising peptide analogues, which exhibit the immunological properties of the peptides described above, but which contain one or more chemical modifications. Such peptide analogues, also referred to as peptide mimetics, can e.g. consist of units corresponding to the amino acid residues of the peptides described above, wherein essentially the same side groups are present, but wherein the backbone contains modifications such as substitution of an amide group (CO—NH) by another group such as CH═CH, CO—O, CO—CH₂ or CH₂—CH₂. Other modifications, such as substitutions of an amino acid by a similar natural, or non-natural amino acid are also envisaged. In this respect, “similar” means having about the same size, charge and polarity; thus the aliphatic amino acids alanine, valine, norvaline, leucine, isoleucine, norleucine and methionine can be considered as similar; likewise the basic to neutral polar amino acids such as lysine, arginine, ornithine, citrulline, asparagine and glutamine are similar for the present purpose; the same applies to the acidic to neutral polar aminoacids like asparagine, aspartate, glutamine, glutamate, serine, homoserine and threonine.

The peptides described above may be used as such, or may be coupled to a sequence which enhances their antigenicity or immunogenicity. Such sequences may include parts of toxoids or immunoglobulins. The peptides may also be used as complexes with MHC molecules and/or incorporated in liposomes. The peptides may also be covalently coupled to other molecules or whole cells as a vector for immunostimulation. The peptides may be in the form of monomers, dimers or multimers.

The invention also provides autologous T cells or other cells expressing a T cell receptor, or part thereof, from such T cells, activated by immunostimulation using a protein and/or peptide as described above.

The invention also concerns antibodies, in particular monoclonal antibodies directed at the protein and/or peptides described above. The antibodies can be produced using known methods, e.g. by hybridoma technology. The antibodies may be used as a passive vaccine or as a diagnostic tool.

### Methods for Identifying HSP70 Epitopes and Peptides

In a further embodiment a method for identifying HSP70-derived peptides suitable for the treatment and/or prevention of one or more IBDs is provided, comprising:

Providing one or more HSP70-derived peptides (“test” peptide), preferably from a bacterial and/or mammalian HSP70 protein; and
a) optionally determining whether the HSP70-derived peptide(s) is/are capable of binding MHC class II molecules, i.e. binding the MHC class II cleft, or being elutable from the MHC class II molecule;
b) optionally testing the capacity of the “test”-peptide(s) to induce peptide specific T-cell which are cross-reactive with the homologous self-peptide of a mammal (especially a human or animal model or cell lines) in a cross-reactivity assay and selecting those peptides which do show cross-reactivity; and
c) optionally administering a composition comprising or consisting of one or more of the “test” peptides in an animal model of an IBD disease to determine the in vivo protective activity, and comparing disease development and/or symptoms between treated and control animals, thereby verifying that the one or more peptides are suitable for treatment and/or prevention of one or more IBDs, and
d) using the one or more “test” peptides for the preparation of a medicament for the treatment and/or prevention of IBDs in humans or animals and in a method for treating and/or preventing IBDs in subjects, especially human or animal subjects, such as farm animals or companion animals.

In step (a) any fragment of a bacterial or mammalian HSP70 protein (including human HSP70 proteins) may be used. Many HSP70 amino acids sequences are available and peptides consisting of or comprising a fragment can be synthesized using standard peptide synthesis methods. Preferably, HSP70 proteins are aligned, e.g. from different bacterial origins and/or mammalian origins, and conserved amino acid regions are chosen. The peptide is referred to as the “test” peptide herein below.

In step (b) one preferably determines whether the “test”—peptide can bind the MHC class II molecule of humans. The capability to bind can be analysed using various methods, for example using an MHC class II-peptide binding assays.

Steps (a) and (b) can also be interchanged, so that initially test peptides are identified which bind MHC class II molecules, for example by literature analysis, and then synthesizing the test peptide(s) for further use in steps (c), (d) and (e). As step (b) is optional, it can also be omitted.

The optional step (c) involves the analysis whether or not the test peptide(s) is/are able to activate regulatory T cells which are cross-reactive with the homologous self peptide or protein comprising the homologous self amino acids. This can be done by providing the homologous self peptide or protein (e.g. by aligning the “test” peptide of step (a) with the native HSP70 protein of the organism or cell line), and by then testing whether administration of the “self” peptide or protein to the T cells which were primed with the “test” peptide, leads to a T cell activation response, such as proliferation, presentation of activation markers and production of cytokines. This can for example be done in vivo (animal models) as well as in vitro (using an in vitro T cell sensitization assay, see Halder T, Pawelec G, Kirkin A F, Zeuthen J, Meyer H E, Kun L, Kalbacher H., Isolation of novel HLA-DR restricted potential tumor-associated antigens from the melanoma cell line FM3., Cancer Res., 1997, 57:3238-44).

Test peptides which show cross-reactivity are selected for further use, while peptides which do not show cross-reactivity are discarded.

In step (d) an animal model of an IBD is used, together with appropriate controls, to determine whether the administration of one or more amounts of the peptide(s) at one or more time points (prior to disease induction or after disease induction) have any effect on disease severity or development. Such animal models of IBD are available in the art, see e.g. the Colitis models described in the Examples. Other IBD models include for example chemically induced, immunological, genetic and spontaneous models (see Pizarro T T, Arseneau K O, Bamias G, Cominelli F., Mouse models for the study of Crohn's disease. Trends Mol Med. 2003 May; 9(5):218-22. Review).

The animal model can also be used to determine what the effective concentration and optimal administration mode and administration regime is.

It is also possible to test the functionality and/or effective amounts of one or more HSP70 derived peptides and/or proteins by using transgenic rodents, such as mice, which have been transformed with human MHC class II genes, see for example Koehm S, Slavin R G, Hutcheson P S, Trejo T, David C S, Bellone C J., HLA-DRB1 alleles control allergic bronchopulmonary aspergillosis-like pulmonary responses in humanized transgenic mice, J Allergy Clin Immunol. 2007, June 8^{th} e-publication ahead of print; Chen Z, de Kauwe A L, Keech C, Wijburg O, Simpfendorfer K, Alexander W S, McCluskey J., Humanized transgenic mice expressing HLA DR4-DQ3 haplotype: reconstitution of phenotype and HLA-restricted T-cell responses, Tissue Antigens 2006 No. 68(3): 210-9; Brintnell W, Bell D A, Hill J A, Jevnikar A M, Sette A, Sidney J, Doege K, Cairns E., The influence of MHC class II molecules containing the rheumatoid arthritis shared epitope on the immune response to aggrecan G1 and its peptides, Scand J Immunol. 2007 Nr. 65(5): 444-52; and Mangalam A, Rodriguez M, David C., Role of MHC class II expressing CD4+ T cells in proteolipid protein(91-110)-induced EAE in HLA-DR3 transgenic mice, Eur J. Immunol. 2006 Nr. 36(12): 3356-70.

In step (e) those peptides or mixtures of peptides which showed a positive effect on treatment and/or prevention of the disease symptoms or progression in step (d) are used to make compositions for clinical or animal trials and for commercial use.

The method may also be used for full lengths HSP70 proteins, or mixtures thereof, or mixtures between peptides and full lengths HSP70 proteins. In the above method step (c) can be done also with full length HSP70 protein(s), thus in vitro sensitization assay followed by measuring cross-reactivity with purified human hsp70 or stressed antigen presenting cells or with human hsp70 derived peptides. It can also be done in animal models. Cross-reactive T cell responses can then be analysed for induction of regulatory activities (e.g. cytokine like IL-10 production, suppressive activity of hsp specific T cells in co-culture assays).

### Compositions and Uses According to the Invention

The proteins and/or peptides of the present invention are used to make pharmaceutical compositions comprising these, whereby the pharmaceutical compositions are useful for administration to mammals, particularly humans and/or animals, especially farm animals or companion animals, to treat and/or prevent IBD (especially Crohn's disease, granulomatous colitis, ulcerative colitis, lymphocyte colitis, collagenous colitis and/or Coeliac disease), or one or more (preferably all) symptoms thereof.

The amount of protein or peptide to be used may vary, depending on whether the composition is for the treatment or for the prophylaxis, and depending on the dosage form and frequency of administration. Suitable formulations are found in Remington's Pharmaceutical Sciences, Mack Publishing Company, Philadelphia, Pa., 18th ed. (1990), or in Remington: The Science and Practice of Pharmacy, 2005, Lippincott Williams & Wilkins, US; 21^{st} Rev Ed edition.

In one embodiment the immunogenic proteins and/or peptides of the invention (or compositions comprising these) are administered prophylactically (prevention) or to an individual already suffering from IBD (treatment). The compositions are administered to a subject (e.g. a patient) in an amount sufficient to elicit an effective immune response. An amount adequate to accomplish this is defined as “therapeutically effective dose” or “immunogenically effective dose”. Amounts effective for this use will depend on, e.g., the protein and/or peptide composition, the manner of administration, the stage and severity of the disease being treated, the weight and general state of health of the subject, and the judgement of the prescribing physician, but generally range for the from about 0.1 μg to about 150 μg per kilogram (kg) of body weight per subject, or from about 1 μg to about 200 μg per kilogram (kg) of body weight, more commonly from about 1 μg to about 50 μg per kg of body weight per dose. A dose may be administered once a week, or once every other day or daily or even several times per day. Dosage units may be administered over a short period (e.g. a few weeks to months) or over longer time periods (several months to years).

The composition may be made in various dosage units, such as doses comprising e.g. 7 μg, 7.5 μg, 8 μg, 9 μg, 10 μg, 20 μg, 30 μg, 40 μg, 50 μg, 100 μg, 200 μg, 1000 μg, 2500 μg, 5000 μg or more protein and/or peptide.

Preferably, the pharmaceutical compositions are administered enteral, most preferably oral. However, in another embodiment other forms of administration are included, such as transdermal, nasal, inhalation, and parenteral. Especially preferred are oral and nasal formulations.

The proteins and/or peptides according to the invention may, for example, be dissolved or suspended in an acceptable carrier, preferably an aqueous carrier. A variety of aqueous carriers may be used, e.g., water, buffered water, 0.4% saline, 0.3% glycine, hyaluronic acid and the like. These compositions may be sterilized by conventional, well-known sterilization techniques, or may be sterile filtered. The resulting aqueous solutions may be packaged for use as is, or lyophilized, the lyophilized preparation being combined with a sterile solution prior to administration.

The compositions may contain pharmaceutically acceptable auxiliary substances as required to approximate physiological conditions, such as buffering agents, tonicity adjusting agents, wetting agents and the like, for example, sodium acetate, sodium lactate, sodium bicarbonate, sodium chloride, potassium chloride, calcium chloride, sorbitan monolaurate, and triethanolamine oleate.

For solid compositions, conventional nontoxic solid carriers may be used which include, for example, pharmaceutical grades of mannitol, lactose, starch, magnesium stearate, sodium saccharin, talcum, cellulose, glucose, sucrose, magnesium carbonate, and the like. For oral administration, a pharmaceutically acceptable nontoxic composition is formed by incorporating any of the normally employed excipients, such as those carriers previously listed, and generally 10-95% of active ingredient, that is, one or more proteins and/or peptides of the invention, and more preferably at a concentration of 25%-75%. As noted above, the compositions are intended to induce an immune response to the peptides. Thus, compositions and methods of administration suitable for maximizing the immune response are preferred. For instance, peptides may be introduced into a host, including humans, linked to a carrier or as a homopolymer or heteropolymer of active peptide units. Alternatively, the a “cocktail” of proteins and/or peptides can be used. A mixture of more than one protein and/or peptide has the advantage of increased immunological reaction.

The compositions, especially oral dosage forms, may further comprise one or more protease inhibitors. Protease inhibitors are divided into four classes: serine protease inhibitors (including trypsin inhibitors), cysteine protease inhibitors, aspartic protease inhibitors, and metalloproteinase inhibitors. Suitable protease inhibitors are available in the art (e.g. from Sigma-Aldrich). A preferred inhibitor is a trypsin inhibitor, such as a plant derived trypsin inhibitor (soybean trypsin inhibitor, lima bean trypsin inhibitor, corn trypsin inhibitor, etc.) or animal derived trypsin inhibitor (trypsin inhibitor from chicken or turkey egg white, from bovine pancreas, etc).

The compositions may also include an adjuvant. A number of adjuvants are well known to one skilled in the art. Suitable adjuvants include incomplete Freund's adjuvant, alum, aluminium phosphate, aluminium hydroxide, N-acetyl-muramyl-L-threonyl-D-iso-glutamine (thr-MDP), N-acetyl-nor-muramyl-L-alanyl-D-isoglutamine (CGP 11637, referred to as nor-MDP), N-acetylmuramyl-L-alanyl-D-isoglutaminyl-L-alanine-2-(1′-2′-dipalmitoyl-sn-glycero-3-hydroxyphosphoryloxy)-ethylamine (CGP 19835A, referred to as MTP-PE), and RIBI, which contains three components extracted from bacteria, monophosphoryl lipid A, trehalose dimycolate and cell wall skeleton (MPL+TDM+CWS) in a 2% squalene/Tween 80 emulsion.

The concentration of immunogenic peptides of the invention in the pharmaceutical formulations can vary widely, i.e. from less than about 0.1% (e.g. 0.01% wt/vol) to about 2% or more by weight, and will be selected primarily by fluid volumes, viscosities, etc., in accordance with the particular mode of administration selected.

Further guidance regarding formulations that are suitable for various types of administration can be found in Remington's Pharmaceutical Sciences, Mace Publishing Company, Philadelphia, Pa., 18th ed. (1990, supra). For a brief review of methods for drug delivery, see, Langer, Science 249:1527-1533 (1990). Both of these references are incorporated herein by reference in their entirety.

Transdermal delivery systems include patches, gels, tapes and creams, and can contain excipients such as solubilizers, permeation enhancers (e.g. fatty acids, fatty acid esters, fatty alcohols and amino acids), hydrophilic polymers (e.g. polycarbophil and polyvinyl pyrollidine and adhesives and tackifiers (e.g. polyisobutylenes, silicone-based adhesives, acrylates and polybutene) Transmucosal delivery systems include patches, tablets, suppositories, pessaries, gels, and creams, and can contain excipients such as solubilizers and enhancers (e.g. propylene glycol, bile salts and amino acids), and other vehicles (e.g. polyethylene glycol, fatty acid esters and derivatives, and hydrophilic polymers such as hydroxypropylmethyl cellulose and hyaluronic acid). Injectable delivery systems include solutions, suspensions, gels, microspheres and polymeric injectables, and can comprise excipients such as solubility-altering agents (e.g. ethanol, propylene glycol and sucrose) and polymers (e.g. polycaprylactones, and PLGA's). Implantable systems include rods and discs, and can contain excipients such as PLGA and polycapryl lactone. Other delivery systems that can be used for administering the pharmaceutical composition of the invention include intranasal delivery systems such as sprays and powders, sublingual delivery systems and systems for delivery by inhalation. For administration by inhalation, the pharmaceutical compositions of the present invention are conveniently delivered in the form of an aerosol spray presentation from pressurised packs or a nebuliser, with the use of a suitable propellant, e.g., dichloro-difluoromethane, trichlorofluoromethane, dichlorotetrafluoroethane, carbon dioxide or other suitable gas. In the case of a pressurised aerosol the dosage unit may be determined by providing a valve to deliver a metered amount. Capsules and cartridges of, e.g., gelatin for use in an inhaler or insufflator may be formulated containing a powder mix of the peptides of the invention and a suitable powder base such as lactose or starch. The pharmaceutical compositions of the invention may be further formulated for administration by inhalation as e.g. described in U.S. Pat. No. 6,358,530. Also other vaccination or administration methods may be used, such as particle bombardment (biolistics), whereby DNA or RNA encoding the epitope(s), i.e. the peptides and/or proteins according to the invention, is coated onto gold particles and these are used to bombard the subjects tissue, see e.g van Drunen et al. Methods Mol Med. 2006; 127:91-105; Gaffal et al. Eur J. Cell Biol. 2006, Available online 22 Aug. 2006) or liposome delivery. Thus, in one embodiment, DNA or RNA encoding the proteins and/or peptides according to the invention are provided (e.g. plasmids comprising the DNA or RNA), as are compositions comprising these.

In another aspect the invention relates to a method for producing a pharmaceutical composition comprising the proteins and/or peptides of the invention. The method comprises at least the steps of mixing the proteins and/or peptides of the invention with a pharmaceutically acceptable carrier and further constituents like adjuvant as described above. Also gold particles coated with one or more peptides and/or proteins according to the invention are provided, for use in biolistic applications.

Also provided is a method for the treatment or prevention of inflammatory bowel diseases (IBD) in a human, comprising administering to a person in need thereof a therapeutically or prophylactically effective amount of a protein and/or peptide as described above. In a preferred embodiment the administration is orally and/or nasally, at a regular interval.

It is noted that the therapeutic and prophylactic (protective) treatments described herein are not limited to the complete abolishment or complete prevention of disease, but in one embodiment also refer to a significant reduction in severity of one or more IBD symptoms in the treated subject group compared to the control group, as described in the examples. For example one or more symptoms associated with the IBD, such as the weight loss (clinical state), colon shortening, and/or histomorphological changes etc. may be significantly reduced in the treated group. A significant reduction should be statistically significant, and the skilled person can easily determine whether this is the case. For example, a reduction in one or more symptoms by at least about 1%, 2%, 5%, 10%, 20% or more, compared to the control group, may be significant.

The following non-limiting Examples describe the protective and therapeutic use of antigenic proteins and peptides of the invention. Unless stated otherwise in the Examples, all molecular techniques are carried out according to standard protocols as described in Sambrook and Russell (2001) Molecular Cloning: A Laboratory Manual, Third Edition, Cold Spring Harbor Laboratory Press, NY, in Volumes 1 and 2 of Ausubel et al. (1994) Current Protocols in Molecular Biology, Current Protocols, USA and in Volumes I and II of Brown (1998) Molecular Biology LabFax, Second Edition, Academic Press (UK).

### Sequences

SEQ ID NO 1: Mycobacterial HSP70 peptide “B1”
SEQ ID NO 2: mouse HSP70 homologue peptide “mB1”
SEQ ID NO 3: Mycobacterial HSP70 peptide “C1”
SEQ ID NO 4 and 5: Mouse HSP70 homologue peptide “mC1a” and “mC1b”
SEQ ID NO 6-16: Mycobacterial HSP70 peptides used in the Examples
SEQ ID NO 17-47: list of MHC class II presented mammalian HSP70 peptides
SEQ ID NO 48-54: human HSP70 proteins
SEQ ID NO 55: Mycobacterium HSP70 protein

### FIGURE LEGENDS

FIG. 1—T-cell proliferation of Example 1 (HSP70 epitope mapping)

FIG. 2—Cytokine production of Example 1 (HSP70 epitope mapping)

FIG. 3—a) and b) Murine DSS colitis experimental schedules

FIG. 4 to FIG. 7—Suppressive effect of full length Mycobacterial HSP70 protein on DSS Colitis (Example 3); FIG. 4 (colon length—peroral group), FIG. 5 (weight change—peroral group), FIG. 6 (Clinical state—peroral group) and FIG. 7 (histological grade—peroral group).

FIG. 8 to FIG. 11—Suppressive effect of full length Mycobacterial HSP70 protein on DSS Colitis (Example 4); FIGS. 8 (Colon length—peroral group), FIG. 9 (weight change—peroral group), FIG. 10 (Clinical state—peroral group) and FIG. 11 (histological grade—peroral group).

FIG. 12—Murine DSS colitis experimental schedule

FIG. 13—Disease onset of Example 5

FIG. 14—Maximum body weight loss of Example 5

FIG. 15—Colon length data of Example 5

FIG. 16—IL10 release from distal colon samples of Example 5

FIG. 17—Murine TNBS colitis experimental schedule

FIG. 18—Effects on body weight of Example 6

FIG. 19—Effects on IL10 production by draining lymphnode cells (CLN); (CD3/CD28 restimulation).

### EXAMPLES

### Example 1

### Mapping of Bacterial HSP70 Peptides Recognized by HSP70 Specific T Cells, Including T Cells that Cross-React with self HSP70

HSP70-specific regulatory T cells, from HSP immunized mice, are a limited source of regulatory T cells (Tregs). Besides that, HSP-specificity is depending on immunization and will be variable. To achieve an alternative source, and a more precisely defined population of Hsp-specific regulatory T cells we started HSP70 epitope mapping.

Spleen cells from Balb/c mice immunized with whole Mycobacterial HSP70 (SEQ ID NO: 55 (intraperitoneally and subcutaneously a total of 100 μg in 200 μl 10 mg/ml dimethyl dioctadecyl ammonium bromide (DDA) as adjuvant in PBS), were analyzed for T cell responses against a complete set of 123 overlapping 15-mer peptides covering the whole protein according to procedures previously described (Wendling et al. (2000) J. Imm. 164: 2711-2717).

In this way we identified 13 Hsp70 peptides that were more or less recognized by Hsp70 specific T cells and possibly involved in activation of Hsp-specific regulatory T cells.

To define the epitopes more precisely, we divided the 13 peptides, based on the degree of sequence identity between bacterial and the homologous mouse Hsp70s, in three pools (pool A: non-conserved peptides, pool B: conserved peptides comprising at least about 80% amino acid identity to the mouse homologue, and pool C: highly conserved peptides comprising >86% amino acid identity to the mouse homologue, see below) and immunized 10-12 weeks old Balb/c mice twice with a 2 week interval (intraperitoneally and subcutaneously) with the peptide pools. 10 days after the second immunization primed spleen and draining lymph node cells were re-stimulated in vitro with all the 13 individual peptides after which we measured T-cell proliferation (tritium incorporation), IFNγ, IL10, IL4 and IL5 production (cytokine production).

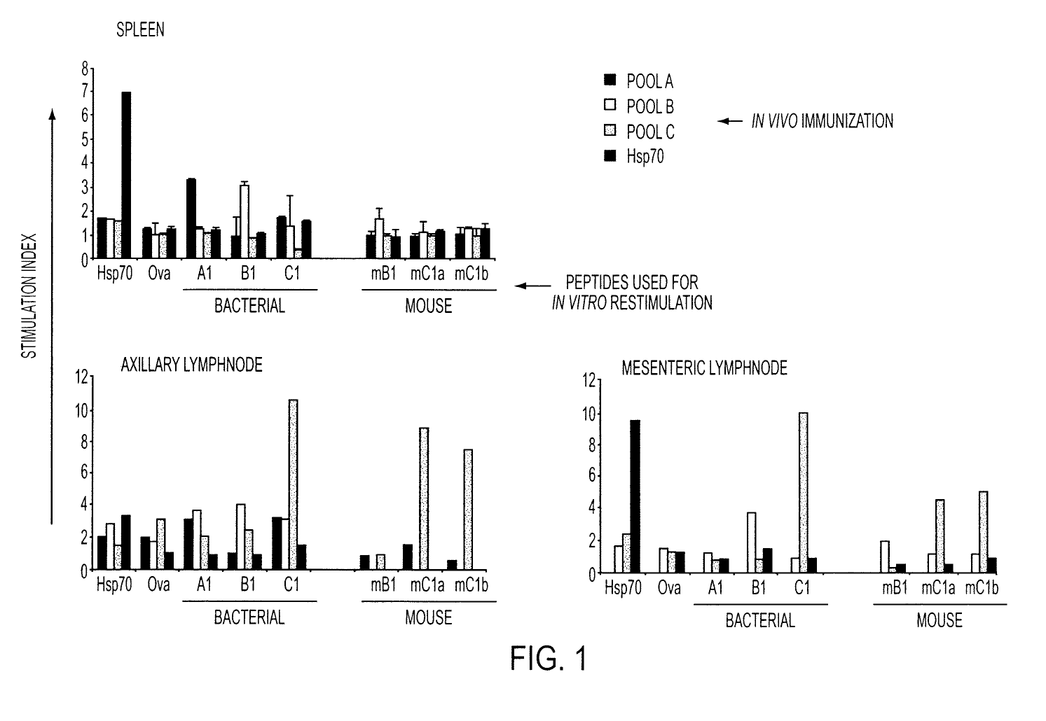

We identified three (out of the thirteen) peptides that induced clear responses (see FIG. 1): three peptides induced increased proliferative T-cell responses and IFNγ (peptides A1, B1, C1) and two peptides induced augmented IL-10 production (peptide A1, C1). None of the peptides induced detectable IL4 or IL5.

Cross reactivity was determined with the corresponding mouse homologue peptides Peptides B1 and C1 were found to have induced cross reactive T cell responses to their corresponding mouse homologue peptides (with respect to both proliferation and cytokine production (see responses to mouse homologue peptides mB1, mC1a, mC1b).

The T cell epitope containing peptides are used to generate peptide specific T cell lines and hybridomas. These T cell lines can be used both in vitro and in vivo as a source of Hsp specific regulatory T cells. Furthermore, activation of the lines and hybridomas is used to study the effect of Hsp manipulation of antigen presenting cells on Hsp peptide specific T-cell activation. Peptides C1 and mC1a have been used in the TNBS colitis studies.

| pool A (not conserved) | pool B (conserved) | pool C (highly conserved) |
|---|---|---|
| A1 = KPFQSVIADTGISVS | B1 = DEVVAVGAALQAGVL | C1 = VLRIVNEPTAAALAY |
| (SEQ ID NO: 6) | (SEQ ID NO: 1) | (SEQ ID NO: 3) |
| A2 = YTAPEISARILMKLK | B2 = EGSRTTPSIVAFARN | C2 = ILVFDLGGGTFDVSL |
| (SEQ ID NO: 7) | (SEQ ID NO: 10) | (SEQ ID NO: 14) |
| A3 = AEGGSKVPEDTLNKV | B3 = MQRLREAAEKAKIEL | C3 = RGIPQIEVTFDIDAN |
| (SEQ ID NO: 8) | (SEQ ID NO: 11) | (SEQ ID NO: 15) |
| A4 = AQAASQATGAAHPGG | B4 = GGKEPNKGVNPDEVV | C4 = QIEVTFDIDANGIVH |
| (SEQ ID NO: 9) | (SEQ ID NO: 12) | (SEQ ID NO: 16) |
| | B5 = LDVTPLSLGIETKGG | |
| | (SEQ ID NO: 13) | |

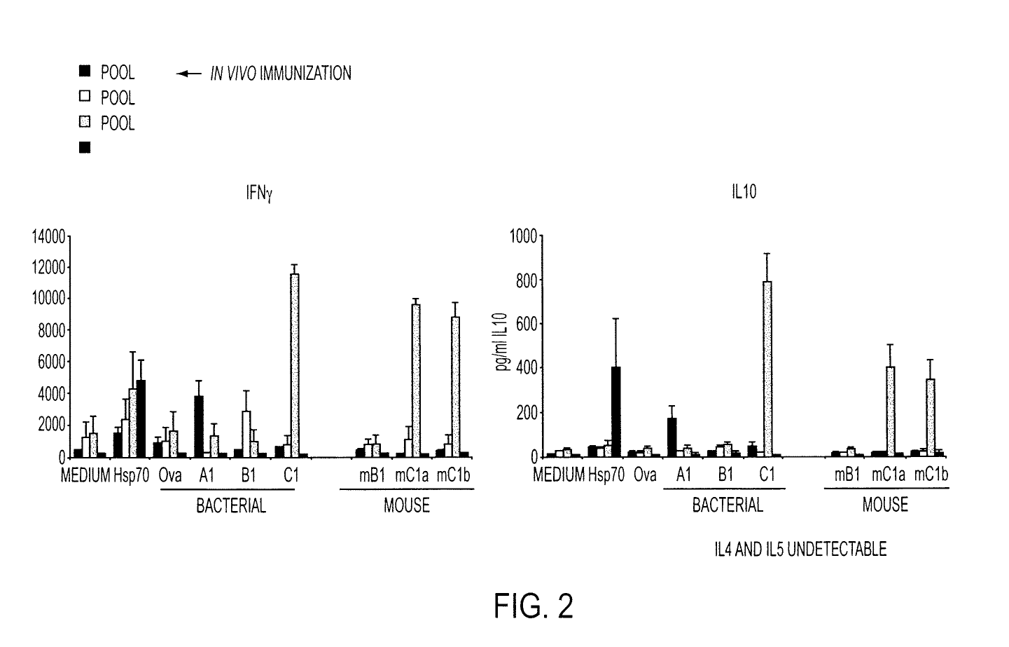

Results are shown in FIGS. 1 and 2, and are summarized below:

TABLE 1
Summary Hsp70 epitope mapping

| | | | | Cross- |
|---|---|---|---|---|
| | | | | reactivity of |
| | | | | bacterially |
| | | | | primed T-cells |
| | T-cell | | | with mouse |
| | proliferation | | | homologue |
| Peptide | response | IFN-gamma | IL-10 | peptide |
| A1 | + | + | + | no |
| (SEQ ID NO: 6) | | | | |
| B1 | + | + | No | Yes |
| (SEQ ID NO: 1) | | | | |
| C1 | ++ | ++ | ++ | yes |
| (SEQ ID NO: 3) | | | | |

Mycobacterial peptides:
A1 = mycobacterial hsp70 peptide

| | |
|---|---|
| (KPFQSVIADTGISVS)- | SEQ ID NO: 6 |

B1 = mycobacterial hsp70 peptide

| | |
|---|---|
| (DEVVAVGAALQAGVL)- | SEQ ID NO: 1 |

C1 = mycobacterial hsp70

| | |
|---|---|
| (VLRIYNEPTAAALAY)- | SEQ ID NO: 3 |

Mouse homologues:
mB1 = mouse Grp75 peptide (hspa9a)

| | |
|---|---|
| (DEAVAIGAAIQGGVL)- | SEQ ID NO: 2 |

mC1a = mouse Grp75 (hspa9a) peptide

| | |
|---|---|
| (VLRVINEPTAAALAY)- | SEQ ID NO: 4 |

mC1b = mouse hsp70 (hspa1a/hspa8) peptide

| | |
|---|---|
| (VLRIINEPTAAAIAY) | SEQ ID NO: 5 |

### Conclusions

The above shows that conserved HSP70 peptides, e.g. B1 and C1 and their mammalian homologues, induce a T cell response and that the bacterially primed T cells cross react with self HSP70 peptides/proteins in the mammal (mouse). Also cytokine production (IFN gamma and IL-10 production) is induced, also by the corresponding mouse homologue peptides.

### Example 2

### The DSS Model and the TNBS Model of Colitis

### The DSS Model of Colitis

A model of colitis that is at least partially related to a change in epithelial cell barrier function is the colitis induced by the physical agent, dextran sodium sulphate (DSS). This model has been frequently used to study the efficacy of potential therapeutic agents because of its ease to induce via administration of DSS in drinking water and because DSS induces a consistent level of colitis with a defined onset. The mechanisms of inflammation in this form of colitis are, at least initially, the activation of nonlymphoid cells such as macrophages and the release of pro-inflammatory cytokines. Changes in epithelial barrier function can be found early (several days before the onset of frank inflammation) and thus may set the stage for macrophage activation.

In the acute stages of DSS colitis the T cell response consists of a polarized Th1 response, but in later and more chronic phases of the inflammation, a mixed Th1/Th2 response occurs. In either case, DSS elicits the secretion of large amounts of TNF-alpha and IL-6, which are mainly responsible for the tissue damage in the disease. The protocols used were adapted from Verdu E F et al, Clin Exp Immunol. 2000, 120:46-50.

### The TNBS Model of Colitis

The TNBS model of colitis can be carried out in multiple ways. One of the best and most representative variant for studying the role of the adaptive immune responses in IBD is the one used here, which is a protocol of skin sensitization followed by intracolonic instillation with TNBS (Te Velde A A, Verstege M I, Hommes D W. Inflamm. Bowel Dis. 2006, 12:995-999. Our protocol was a modification (adaptation to local conditions) of the protocol described by Arita M et al. (Proc Natl Acad Sci USA. 2005, 102:7671-6).

### Example 3

### Suppressive Effect of Administration of Full Length Mycobacterial HSP70 Protein on DSS Colitis in BALB/c Mice (Prague study—1^{st} Part)

This is a first part of one experiment. Due to animal number limitations the experiment was split up in two parts. In each part there were five mice per group.

### Protocol

Fifteen conventional female Balb/c female mice (7 to 9 weeks old) were randomly separated in 4 groups (five mice each). To evaluate the efficacy of per oral treatment, the antigens (full length Mycobacterial HSP70 protein) were administered together with 1 mg of Soybean trypsin inhibitor (SBTI, Sigma) dissolved in 50 μl of 0.15 M sodium bicarbonate, by gavage 4 times in one week interval. After one week the mice received 3% dextran sodium sulfate (DSS) in their drinking water continuously for up to 7 days. Colitis was evaluated on day 35 by a clinical activity score, by colon length and histological score.

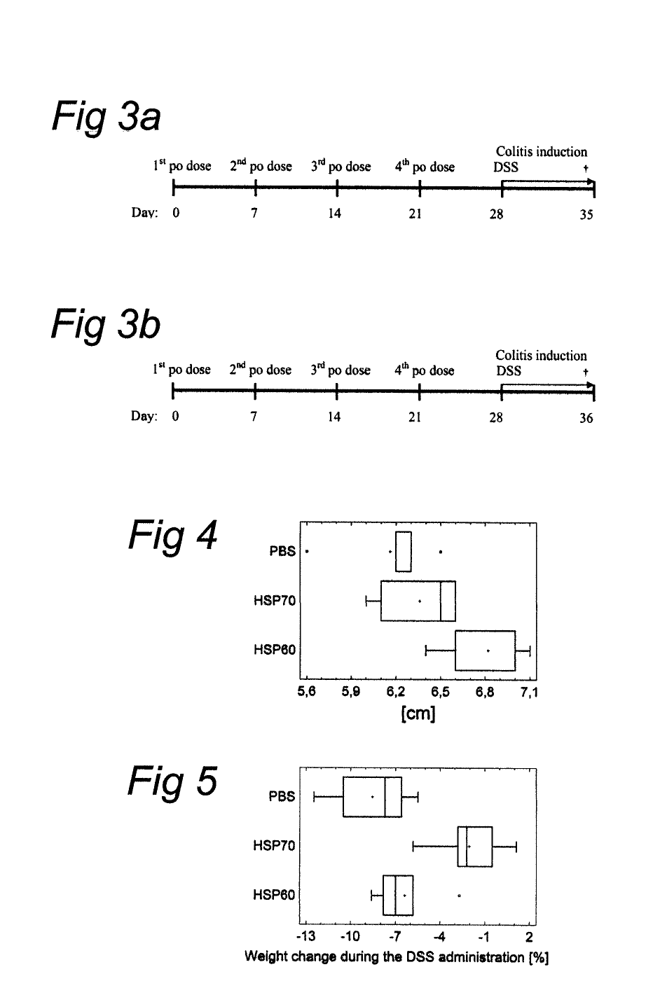

For the experiment schedule, see FIG. 3 (schedule A).

### GROUP A1 (PBS+DSS):

treated: 100 μl of sterile PBS/SBTI intragastrically (Control group)

### GROUP B1 (HSP60+DSS):

treated: HSP60 30 μg/mouse in 100 μl of sterile PBS/SBTI (1:1) intragastrically

### GROUP C1 (HSP70+DSS):

treated: HSP70 30 μg/mouse in 100 μl of sterile PBS/SBTI (1:1) intragastrically

### Results

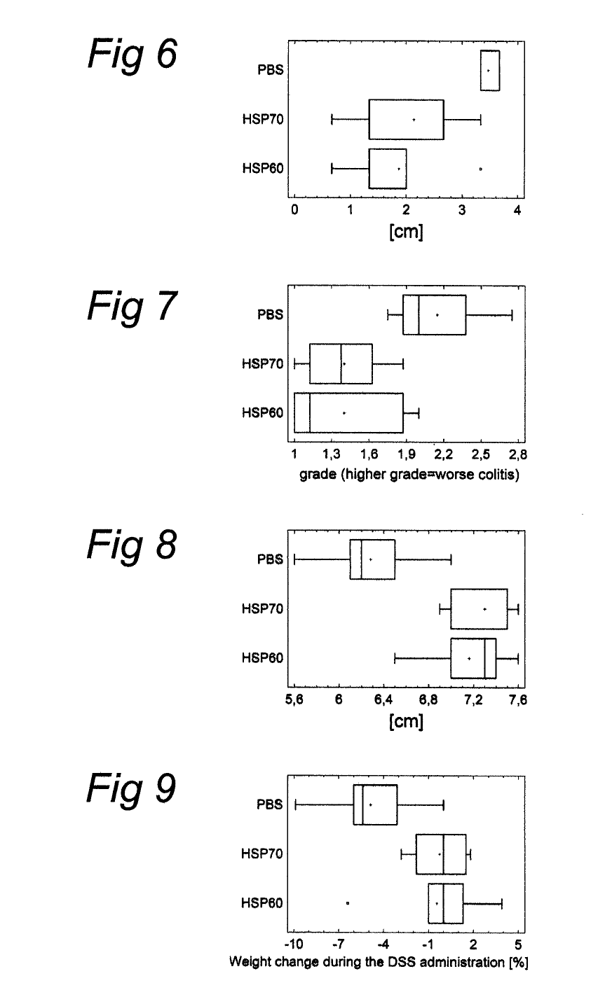

Results are shown in the tables below and in FIG. 4 (colon length), FIG. 5 (weight change), FIG. 6 (Clinical state) and FIG. 7 (histological grade).

### Colon Length:

Summary Statistics

| | | | | Standard | | | |
|---|---|---|---|---|---|---|---|
| | Count | Average | Variance | deviation | Minimum | Maximum | Range |
| PBS | 5 | 6.16 | 0.113 | 0.336155 | 5.6 | 6.5 | 0.9 |
| HSP60 | 5 | 6.82 | 0.092 | 0.303315 | 6.4 | 6.6 | 0.6 |
| HSP70 | 5 | 6.36 | 0.083 | 0.288097 | 6.0 | 7.1 | 0.7 |
| Total | 15 | 6.45 | 0.164 | 0.405087 | 5.6 | 7.1 | 1.5 |

Multiple Range Tests

| Contrast | Difference | p-value | CI 95% |
|---|---|---|---|
| PBS - HSP70 | −0.2 | 0.3420 | −0.656567 to 0.256567 |
| PBS - HSP60 | *−0.66 | 0.0115 | −1.12693 to −0.193069 |
| HSP70 - HSP60 | *−0.46 | 0.0394 | −0.891415 to −0.0285851 |

*denotes a statistically significant diference. (p < 0.05)

### Weight Change

Summary Statistics

| | | | | Standard | | | |
|---|---|---|---|---|---|---|---|
| | Count | Average | Variance | deviation | Minimum | Maximum | Range |
| PBS | 5 | −8.56 | 8.308 | 2.88236 | −12.5 | −5.5 | 7.0 |
| HSP60 | 5 | −6.38 | 5.302 | 2.30261 | −8.6 | −2.7 | 5.9 |
| HSP70 | 5 | −2.04 | 6.743 | 2.59673 | −5.8 | 1.1 | 6.9 |
| Total | 15 | −5.66 | 13.684 | 3.69919 | −12.5 | 1.1 | 13.6 |

Multiple Range Tests

| Contrast | Difference | p-value | CI 95% |
|---|---|---|---|
| PBS - HSP70 | *−6.52 | 0.0056 | −10.5209 to −2.51909 |
| PBS - HSP60 | −2.18 | 0.2229 | −5.98457 to 1.62457 |
| HSP70 - HSP60 | *4.34 | 0.0233 | 0.760853 to 7.91915 |

*denotes a statistically significant diference. (p < 0.05)

### Clinical State:

| Parameter* | | Score |
|---|---|---|
| Weight loss | >5% | 0 |
| | 5-10% | 2 |
| | 10-20% | 3 |
| | >20% | 4 |
| Stool | well formed pellets | 0 |
| consistency | pasty and semiformed stools that don't stick to | 2 |
| | the anus | |
| | liquid stools that did stick to the anus | 4 |
| Bleeding | no blood in hemoccult | 0 |
| | positive hemoccult (Okult viditest rapid, Vidia | 2 |
| | s.r.o.) | |
| | gross bleeding | 4 |

Total score is calculated by adding the score for each parameter and divided by 3.
*Cooper et al.: Clinicopathologic study of dextran sulfate sodium experimental murine colitis. Lab Invest 69: 238-249, 1993, and Hermann et al.: Specific Type IV Phosphodiesterase Inhibitor Rolipram Mitigates Experimental Colitis in Mice JPET 292: 22-30, 2000

Summary Statistics

| | | | | Standard | | | |
|---|---|---|---|---|---|---|---|
| | Count | Average | Variance | deviation | Minimum | Maximum | Range |
| PBS | 5 | 3.4666 | 0.0334668 | 0.182939 | 3.333 | 3.667 | 0.334 |
| HSP60 | 5 | 1.8666 | 0.977422 | 0.988647 | 0.667 | 3.333 | 2.666 |
| HSP70 | 5 | 2.1334 | 1.19987 | 1.09538 | 0.667 | 3.333 | 2.666 |
| Total | 15 | 2.4889 | 1.15648 | 1.0754 | 0.667 | 3.667 | 3.0 |

Multiple Range Tests

| Contrast | Difference | p-value | CI 95% |
|---|---|---|---|
| PBS - HSP70 | *1.3332 | 0.0277 | 0.187908 to 2.47849 |
| PBS - HSP60 | *1.6 | 0.0074 | 0.563121 to 2.63688 |
| HSP70 - HSP60 | 0.4 | 0.6966 | −1.78852 to 1.25492 |

*denotes a statistically significant diference. (p < 0.05)

### Histological Grade

Summary Statistics

| | | | | Standard | | | |
|---|---|---|---|---|---|---|---|
| | Count | Average | Variance | deviation | Minimum | Maximum | Range |
| PBS | 5 | 2.15 | 0.167187 | 0.408886 | 1.75 | 2.75 | 1.0 |
| HSP60 | 5 | 1.4 | 0.128125 | 0.357946 | 1.0 | 1.875 | 0.875 |
| HSP70 | 5 | 1.4 | 0.245312 | 0.49529 | 1.0 | 2.0 | 1.0 |
| Total | 20 | 1.65 | 0.288393 | 0.537022 | 1.0 | 2.75 | 1.75 |

Multiple Range Tests

| Contrast | Difference | p-value | CI 95% |
|---|---|---|---|
| PBS - HSP70 | *0.75 | 0.0150 | 0.189576 to 1.31042 |
| PBS - HSP60 | *0.75 | 0.0311 | 0.0876491 to 1.41235 |
| HSP70 - HSP60 | 0.0 | 1.0000 | −0.63021 to 0.63021 |

*denotes a statistically significant diference. (p < 0.05)

### Conclusions

The above results show that full length HSP70 protein has a suppressive effect on colitis when administered orally, prophylactically (prior to disease induction).

### Example 4

### Suppressive Effect of Full Length Mycobacterial HSP70 on DSS Colitis in BALB/c Mice (Prague Study—2^{nd} Part)

These results address only the second part of the experiment (see Example 3 for first part). This part has five mice per group.

### Protocol

Fifteen conventional female Balb/c female mice (7 to 9 weeks old) were randomly separated in 4 groups (five mice each). To evaluate the efficacy of per oral treatment, the antigens (full length Mycobacterial HSP70 protein) were administered together with 1 mg of Soybean trypsin inhibitor (SBTI, Sigma) dissolved in 50 μl of 0.15 M sodium bicarbonate, by gavage 4 times in one week interval. After one week the mice received 3% dextran sodium sulphate (DSS) in their drinking water continuously for up to 8 days. Colitis was evaluated on day 36 by a clinical activity score, by colon length and histological score.

For the Experimental schedule, see FIG. 3, schedule B.

### Groups

### GROUP A2 (PBS+DSS):

treated: 100 μl of sterile PBS/SBTI intragastrically (Control)

### GROUP B2 (HSP60+DSS):

treated: HSP60 30 μg/mouse in 100 μl of sterile PBS/SBTI (1:1) intragastrically

### GROUP C2 (HSP70+DSS):

treated: HSP70 30 μg/mouse in 100 μl of sterile PBS/SBTI (1:1) intragastrically

### Results

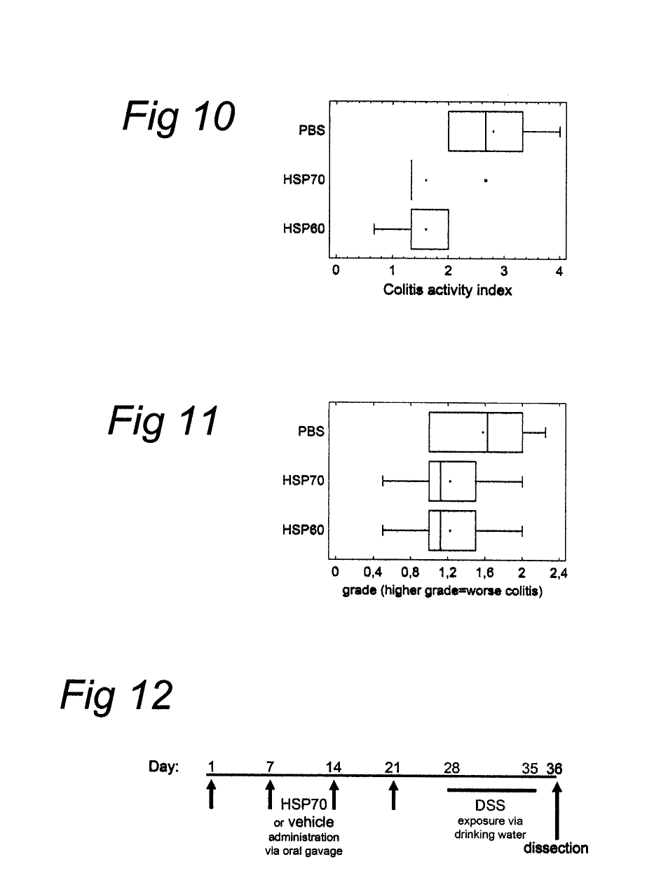

Results are shown below and in FIGS. 8 (Colon length), FIG. 9 (weight change), FIG. 10 (Clinical state) and FIG. 11 (histological grade).

### Colon Length:

HSP70 prevents colon shortening compared to PBS when administered perorally.

Summary Statistics

| | | | | Standard | | | |
|---|---|---|---|---|---|---|---|
| | Count | Average | Variance | deviation | Minimum | Maximum | Range |
| PBS | 5 | 6.28 | 0.267 | 0.51672 | 5.6 | 7.0 | 1.4 |
| HSP70 | 5 | 7.3 | 0.105 | 0.324037 | 6.9 | 7.6 | 0.7 |
| HSP60 | 5 | 7.16 | 0.183 | 0.427785 | 6.5 | 7.6 | 1.1 |
| Total | 15 | 6.91 | 0.377 | 0.613964 | 5.6 | 7.6 | 2.0 |

Multiple Range Tests

| Contrast | Difference | p-value | CI 95% |
|---|---|---|---|
| PBS - HSP70 | *−1.02 | 0.00570944 | −1.649 to −0.391004 |
| PBS - HSP60 | *−0.88 | 0.0189015 | −1.5718 to −0.188197 |
| HSP70 - HSP60 | 0.14 | 0.57574 | −0.693442 to 0.413442 |

*denotes a statistically significant diference. (p < 0.05)

### Weight Change:

Summary Statistics

| | | | | Standard | | | |
|---|---|---|---|---|---|---|---|
| | Count | Average | Variance | deviation | Minimum | Maximum | Range |
| PBS | 5 | −4.88 | 13.427 | 3.66429 | −9.9 | 0.0 | 9.9 |
| HSP60 | 5 | −0.26 | 4.058 | 2.01445 | −2.8 | 1.8 | 4.6 |
| HSP70 | 5 | −0.44 | 14.473 | 3.80434 | −6.4 | 3.9 | 10.3 |
| Total | 15 | −1.86 | 14.0226 | 3.74467 | −9.9 | 3.9 | 13.8 |

Multiple Range Tests

| Contrast | Difference | p-value | CI 95% |
|---|---|---|---|
| PBS - HSP70 | *−4.62 | 0.0386771 | −8.9323 to −0.3077 |
| PBS - HSP60 | −4.44 | 0.0969649 | −9.88726 to 1.00726 |
| HSP70 - HSP60 | 0.18 | 0.927806 | −4.61941 to 4.25941 |

*denotes a statistically significant diference. (p < 0.05)

### Histological Grade:

Summary Statistics

| | | | | Standard | | | |
|---|---|---|---|---|---|---|---|
| | Count | Average | Variance | deviation | Minimum | Maximum | Range |
| PBS | 5 | 1.575 | 0.325 | 0.570088 | 1.0 | 2.25 | 1.25 |
| HSP60 | 5 | 1.225 | 0.316 | 0.561805 | 0.5 | 2.0 | 1.5 |
| HSP70 | 5 | 1.225 | 0.316 | 0.561805 | 0.5 | 2.0 | 1.5 |
| Total | 15 | 1.342 | 0.302 | 0.549892 | 0.5 | 2.25 | 1.75 |

Multiple Range Tests

| Contrast | Difference | p-value | CI 95% |
|---|---|---|---|
| PBS - HSP70 | 0.35 | 0.356811 | −0.475426 to 1.17543 |
| PBS - HSP60 | 0.35 | 0.356811 | −0.475426 to 1.17543 |
| HSP70 - HSP60 | 0.0 | 1.0 | −0.819364 to 0.819364 |

### Conclusions

Administration of Mycobacterial HSP70 protein significantly mitigates the symptoms of colitis compared to PBS (p=0.0057, 0.0387 and 0.0831 for colon length, weight loss and clinical state respectively). The results are similar to those obtained during the first part of the experiment (see Example 3).

### Both Examples 3 and 4 Together

Evaluation of acute dextran sodium sulphate (DSS)-induced colitis in perorally treated BALB/c mice. Values are expressed as means±standard deviations

| | | Colon length | Disease activity | Histological |
|---|---|---|---|---|
| Experimental group | n | (cm) | index | grade |
| PBS/SBTI^{a} | 10 | 6.22 ± 0.42 | 3.13 ± 0.69 | 1.86 ± 0.56 |
| HSP60/SBTI | 10 | 6.99 ± 0.39** | 1.73 ± 0.78** | 1.31 ± 0.51* |
| HSP70/SBTI | 10 | 6.83 ± 0.57* | 1.87 ± 0.88** | 1.31 ± 0.45* |

^{a}Soybean trypsin inhibitor (Sigma-Aldrich), 1 mg/dose dissolved in 50 μl of 0.15 moles/litre sodium bicarbonate buffer (pH 8.0).
*Significantly different (P < 0.05) from value for control group as calculated by Student's t test.
**Significantly different (P < 0.01) from value for control group as calculated by Student's t test.

### Example 5

### Suppressive Effect of Full Length Mycobacterial HSP70 Protein on Induction of DSS Colitis in BALB/c Mice (Utrecht Study)

FIG. 12 shows the study setup. Vehicle refers to PBS.

### Treatment:

### Group A: 100 μl PBS (Control)

Group B: 30 μg Mycobacterial HSP70 protein in 100 μl PBS [SEQ ID NO: 55]

This study is performed with female BALB/c mice aged 7 weeks at the start of the experiment. Groups of mice (n=10) were treated 4 times (days 7, 9, 11, 14) with 30 μg HSP70 in 100 μl PBS (Mt hsp70; batch 031205; 2.1 Endotoxin Units/mg) or vehicle (100 μl PBS), administered via oral gavage. 10 minutes prior to gavage animals received 2 mg soybean trypsin inhibitor (SBTI) in 0.15M sodium bicarbonate, pH 8.0. Colitis was induced by administering drinking water supplemented with 3% DSS during 7 days.

Disease onset was followed by determination of body weight and stool consistency, resulting in clinical state as explained in the table below.

At dissection, colon length was recorded

Colons were cut longitudinally and divided into parts to determine:
histology on formalin-fixed paraffin sections
MPO activity in homogenates (indicative for neutrophilic infiltrations)
Ex-vivo cytokine release (TNFα, IL10) from distal colon sample

### Determination of Clinical Score

| Parameter | | Score |
|---|---|---|
| Body weight loss | <3% | 0 |
| | 3-5% | 1 |
| | 5-10% | 2 |
| | 10-20% | 3 |
| | >20% | 4 |
| Stool consistency | Well formed pellets | 0 |
| | Easy to smear | 1 |
| | Loose stool | 2 |
| | Watery or no stool | 4 |
| Bleeding | No blood | 0 |
| | Blood within faeces | 2 |
| | Gross bleeding | 4 |

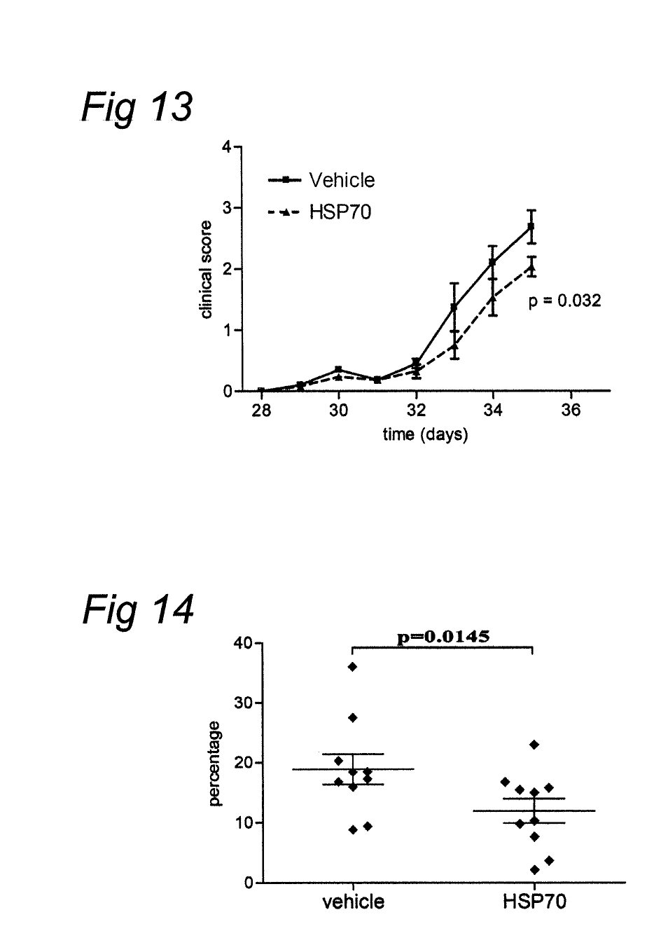

Total score is calculated by the adding the score for each parameter and divided by 3. FIG. 13 shows disease progression. Pre-treatment with HSP70 protein resulted in decreased disease severity. Disease onset is based on clinical score calculated as listed above.

FIG. 14 shows weight loss. HSP70 treatment resulted in significantly less body weight loss due to colitis induction. Maximum body weight loss was determined on day 36.

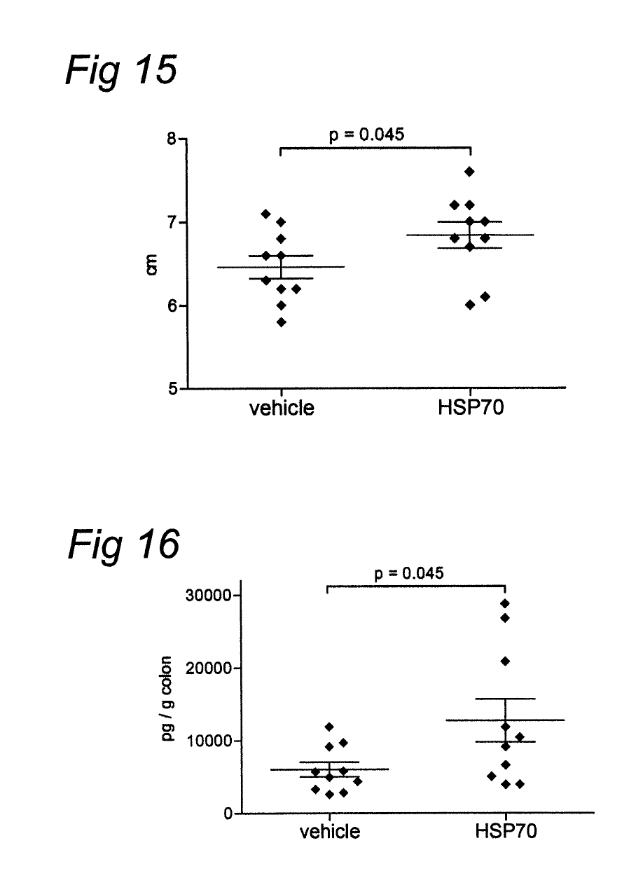

FIG. 15 shows Colon length. Colon length is less decreased upon pretreatment with HSP70 protein. Colonic inflammation normally results in reduced colon length.

FIG. 16 shows that HSP70 treatment increased (interleukin 10) IL10 release. Distal colon samples were incubated for 24 h and IL10 content in supernatant was measured using a specific ELISA. IL10 release from distal colon sample in healthy BALB/c mice is 450±328 pg/g colon.

### Summary of the Results

| | Maximum body weight | | | | |
|---|---|---|---|---|---|
| | loss (percentage) | | | | |
| | | | SEM | | P value |
| treatment | average | st.dev | (n) | day | compared to vehicle |
| Vehicle | −18.88 | 8.00 | 2.53 | 36 | |
| HSP70 | −11.94 | 6.43 | 2.03 | 36 | 0.0145 |

| | | | | P value |
|---|---|---|---|---|
| treatment | average | st. dev | SEM (n) | compared to vehicle |
| | Colon Length (cm) | | | |
| Vehicle | 6.5 | 0.4 | 0.1 | |
| HSP70 | 6.8 | 0.5 | 0.2 | 0.0445 |
| | Colon Weight (g/6 cm) | | | |
| Vehicle | 186 | 18 | 6 | |
| HSP70 | 196 | 22 | 7 | 0.095 |
| | IL10 (pg/g colon) | | | |
| Vehicle | 6028 | 3187 | 1008 | |
| HSP70 | 12742 | 9373 | 2964 | 0.0445 |

Statistics: Non parametric Kruskal Wallis test, followed by Mann-Whitney post-hoc test (1-tailed).

### Example 6

### Suppressive Effect of Full Length Mycobacterial HSP70 Protein, or Mycobacterial and Mouse HSP70 Peptide Homologues, in TNBS Colitis in BALB/c Mice

Aim: to investigate if HSP70 protein or peptide pre-treatment results in attenuated TNBS-induced colitis.

### Study Outline:

Female BALB/c mice (Charles River-Germany), 10-12 weeks of age at start of the experiment, were either treated intragastrically or intranasally with full HSP70 [SEQ ID NO: 55] or HSP70 peptides (Mycobacterial HSP70 derived peptide “C1” shown in SEQ ID NO: 3 and mouse homologue peptide “mC1a” of SEQ ID NO: 4) prior to disease induction. Mice received 4×30 μg HSP70 intragastrically, in a period of 4 weeks. Stomach contents were neutralized by administering soybean trypsin inhibitor (SBTI) 10 minutes prior to oral HSP70 administration.

Intranasal treatment was performed with 4×30 μg HSP70 or 4×(67 μg peptide C1 (Mycobacterial tub. HSP70 peptide 141-155, VLRVNEPTAAALAY) mixed with 33 μg peptide mC1a (=mouse Grp75 (hspa9) peptide 216-230, VLRVINEPTAAALAY)), in a period of 1 week.

Next, mice were sensitized by painting the shaved abdomen with 1 mg of TNBS in 100 μl 50% ethanol on two consecutive days, followed by a rectal challenge with 1 mg TNBS in 100 μl 40% ethanol, after 6 days. Mice were dissected 3 days later, and colonic inflammation was determined based on colon length, weight and morphology. Furthermore, cytokine production within colon and draining caudal lymphnodes (CLN) were determined.

Treatment groups (n=10):
A. Oral PBS, sensitization TNBS, challenge TNBS (control group)
B. Oral (full length) HSP70, sensitization TNBS, challenge TNBS (oral group)
C. Intranasal (full length) HSP70, sensitization TNBS, challenge TNBS (intranasal group)
D. Intranasal peptide, sensitization TNBS, challenge TNBS (peptide group, C1 and mC1a)

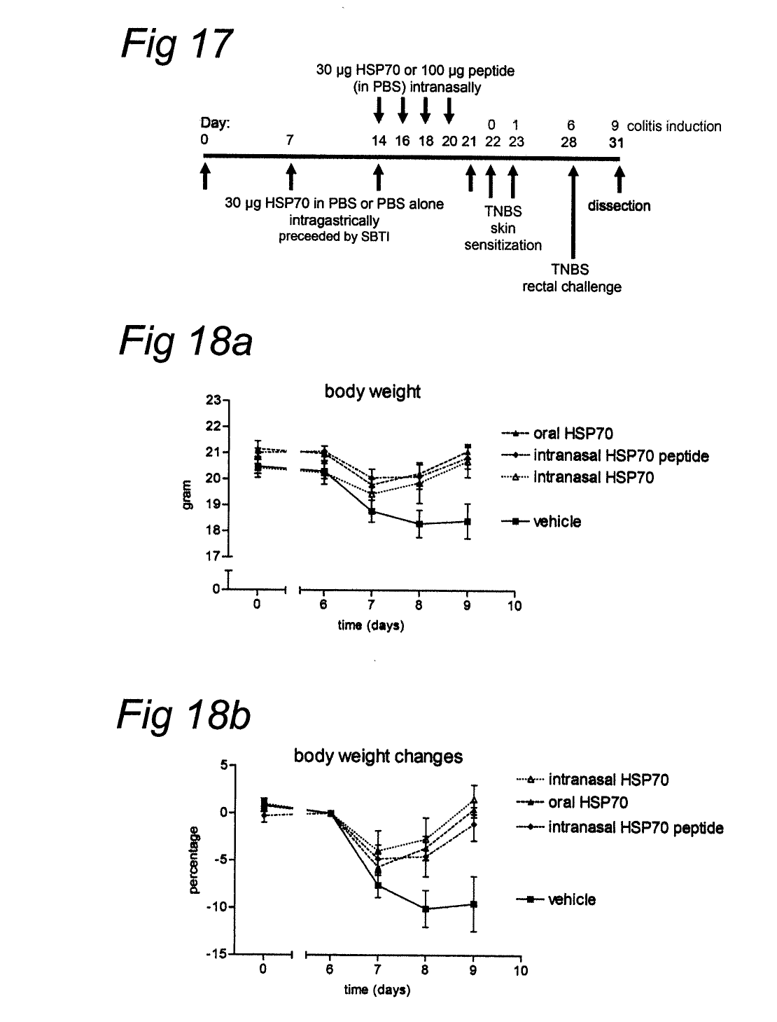

The experimental setup is shown in FIG. 17.

### Results

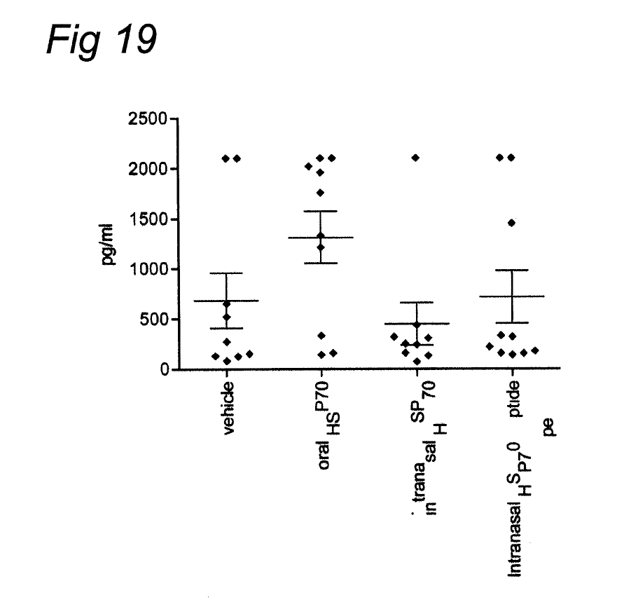

The result on body weight is shown in FIG. 18, and on IL10 production in FIG. 19.

### Conclusions

The above results show that both full length HSP70 protein (either orally or intranasally given) as well as the combination of HSP70 peptides (intranasally given) have a suppressive effect on TNBS induced colitis, prophylactically (prior to disease induction). The suppressive effect of orally given full length HSP70 is associated with elevated IL-10 responses in the caudal lymphnodes.

### Example 7

### List of HSP70 Fragments that Bind MHC class II Molecules

The table below shows the result of a literature study of MHC class II bound/eluted mammalian HSP70 derived peptides. The peptides are useful for the treatment or prevention of one or more IBDs in humans.

TABLE 2
Peptides derived from mammalian HSP70 family members that have been
eluted from MHC Class II molecules

| | MHC class II | MHC | Potential Peptide | Protein | |
|---|---|---|---|---|---|
| Sequence and SEQ ID | type | source | source | source($) | Reference |
| IIANDQGNRTTPSY | I-Ak | mouse | hspa8 (28-41) | m | Nelson et al., |
| (SEQ ID NO: 17) | | | | | (1992), PNAS, |
| | | | | | 89: 7380-83 |
| | | | hspa2 (29-42) | m | |
| | | | hspa1l (30-43) | m | |
| | | | hspa1a (28-41) | m | |
| | | | hspa1b (28-41) | m | |
| ITPSYVAFTPEGERL | I-Ab | mouse | hspa5 (62-76) | m | Dongre et al. |
| (SEQ ID NO: 18) | | | | | (2001), Eur. J. |
| | | | | | Immunol., |
| | | | | | 31: 1485-1494 |
| TPSYVAFTDTERLIG | HLA-DR7 | human | hspa8 (38-52) | h | Chicz et al. |
| (SEQ ID NO: 19) | | | | | (1993), J. Exp. |
| | | | | | Med., 178: 27-47 |
| | | | hspa2 (39-53) | h | |
| | | | hspa1l (40-54) | h | |
| | | | Hspa1a (38-52) | h | |
| TPSYVAFTDTERLIG | HLA-DR7 | human | as above | h | Chicz et al. |
| DA | | | | | (1993), J. Exp. |
| (SEQ ID NO: 20) | | | | | Med. 178: 27-47 |
| DAAKNQLTSNPEN | I-Ag7 | mouse | hspa5 (79-91) | m | Suri et al., |
| (SEQ ID NO: 21) | | | | | (2005), J. Clin. |
| | | | | | Invest., |
| | | | | | 115: 2268-2276 |
| | | | | | (supl) |
| NPTNTVFDAKRLIGR | HLA- | human | hspa8 (62-79) | h | Verreck et al. |
| RFD | DRB1*1104 | | | | (1996) |
| (SEQ ID NO: 22) | | | | | Immunogenetics, |
| | | | | | 43: 392-397 |
| QDIKFLPFKVVEKKT | BoLA- | Bovine | hspa5 (111-128) | m | Sharif et al. |
| KPY | DRB3*1201(in | | | | (2003) Anim. |
| (SEQ ID NO: 23) | mouse line) | | | | Genet., 34: 116-123 |
| LNVLRIINEPTAAAI | HLA- | human | hspa8 (167-184) | r | Muntasell et al. |
| AYG | DRB*0401 | | | | (2004) J. |
| (SEQ ID NO: 24) | (in rat line) | | | | Immunol., |
| | | | | | 173: 1085-1093 |
| (NVLRIINEPTAAAI | | | hspa1a (167-184) | r | |
| AYG) | | | | | |
| (SEQ ID NO: 25) | | | hspa1l (169-186) | r | |
| | | | hspa2 (168-185) | r | |
| NVLRIINEPTAAAIA | HLA- | human | hspa8 (168-184) | h | Dengjel et al., |
| YG | DRB1*0401/ | | | | (2005), PNAS, |
| (SEQ ID NO: 26) | DRB4*0101 | | | | 102: 7922-7927. |
| | | | | | tbl 1 |
| | | | hspa1a (168-184) | h | |
| | | | hspa1l (170-186) | h | |
| | | | hspa2 (169-185) | h | |
| | | | hspa6 (170-186) | h | |
| NVLRIINEPTAAAIA | multiple HLA | human | hspa8 (168-184) | h | Halder et al. |
| (SEQ ID NO: 27) | mix | | | | (1997) Cancer |
| | | | | | Res., 57: 3238-3244 |
| | | | hapa1a (168-184) | h | |
| | | | hspa1l (170-186) | h | |
| | | | hspa2 (169-185) | h | |
| | | | hspa6 (170-186) | h | |
| NVMRIINEPTAAAIA | multiple HLA | human | hspa5 (194-210) | h | Halder et al. |
| YG | mix | | | | (1997) Cancer |
| (SEQ ID NO: 28) | | | | | Res., 57: 3238-3244 |
| VMRIINEPTAAAIAYG | HLA- | human | hspa5 (195-210) | h | Dengjel et al., |
| (SEQ ID NO: 29) | DRB1*0401/ | | | | (2005), PNAS, |
| | DRB4*0101 | | | | 102: 7922-7927 |
| | | | | | (supl. tbl 3) |
| IINEPTAAAIAYGLD | HLA-DQ6 | human | hspa8 (172-186) | h | Sanjeevi et al. |
| (SEQ ID NO: 30) | (B*602) | | | | (2002), Ann. |
| | | | | | N.Y. Acad. Sci., |
| | | | | | 958: 317-320 |
| | | | hspa2 (173-187) | h | |
| | | | hspa1l (174-188) | h | |
| | | | hspa1a (172-186) | h | |
| | | | hspa5 (198-212) | h | |
| NRMVNHFIAEFKRK | I-Ek | mouse | hspa8 (236-249) | m | Marrack et al. |
| (SEQ ID NO: 31) | | | | | (1993) J. Exp. |
| | | | | | Med., 178: 2173-2183 |
| RMVNHFIAEFKRKH | I-Ek | mouse | hspa8 (236-249) | m | Freed et al. |
| (SEQ ID NO: 32) | | | | | (2000), J. |
| | | | | | Immunol., |
| | | | | | 164: 4697-4705 |
| VNHFIAEFKRKHKKD | HLA- | human | hspa8 (238-252) | h | Newcomb and |
| (SEQ ID NO: 33) | DR11/w52 | | | | Cresswell |
| | | | | | (1993), J. Imm., |
| | | | | | 150: 499-507 |
| XDFYTSITRAXFEE | HLA- | human | hspa8 (291-304) | h | Newcomb and |
| (SEQ ID NO: 34) | DR11/w52 | | | | Cresswell |
| | | | | | (1993), J. Imm., |
| | | | | | 150: 499-507 |
| | | | hspa1a (291-304) | h | |
| | | | hspa1l (293-306) | h | |
| | | | hspa2 (294-307) | h | |
| | | | hspa6 (294-306) | h | |
| EGEDFSETLTRAKFE | BoLA- | bovine | hspa5 (315-331) | m | Sharif et al. |
| EL | DRB3*1201(in | | | | (2003) Anim. |
| (SEQ ID NO: 35) | mouse line) | | | | Genet., 34: 116-123 |
| ADLFRGTLDPVEK | HLA-DQ6 | human | hspa8 (307-319) | h | Sanjeevi et al. |
| (SEQ ID NO: 36) | (B*0604) | | | | (2002), Ann. |
| | | | | | N.Y. Acad. Sci., |
| | | | | | 958: 317-320 |
| TIPTKQTQTFTTYSD | RT1.BI | rat | hspa8 (419-436) | r | Reisiz et al. |
| NQP | | | | | (1996) Intern. |
| (SEQ ID NO: 37) | | | | | Immunol., |
| | | | | | 8: 1825-1832 |
| | | | hspa1a (419-436) | r | |
| VPTKKSQIFSTASDN | HLA- | human | hspa5 (443-462) | h | Dengjel et al., |
| QPTVT | DRB1*0401/ | | | | (2005), PNAS, |
| (SEQ ID NO: 38) | DRB4*0101 | | | | 102: 7922-7927 |
| | | | | | (supl. tbl 3) |
| GERAMTKDNNLLG | HLA- | human | hspa8 (445-457) | h | Friede et al., |
| (SEQ ID NO: 39) | DR4Dw4 | | | | (1996), BBA, |
| | | | | | 1316: 85-101 |
| | | | hspa1a (445-457) | h | |
| | | | hspa1l (447-459) | h | |
| | | | hspa2 (448-460) | h | |
| | | | hspa6 (447-459) | h | |
| GERAMTKDNNLLGKFE | HLA- | human | hspa8 (445-460) | h | Dengjel et al., |
| (SEQ ID NO: 40) | DRB1*0401/ | | | | (2005), PNAS, |
| | DRB4*0101 | | | | 102: 7922-7927 |
| | | | | | tbl 1 |
| | | | hspa1a (445-460) | h | |
| GERAMTKDNNLLGRFE | HLA- | human | hspa6 (447-462) | h | Dengjel et al., |
| (SEQ ID NO: 41) | DRB1*0401/ | | | | (2005), PNAS, |
| | DRB4*0101 | | | | 102: 7922-7927 |
| | | | | | (supl. tbl 3) |
| ANGILNVSAVDKSTG | HLA- | human | hspa8 (482-499) | h | Lippolis et al. |
| KE | DRB*0401 | | | | (2002), J. Imm., |
| (SEQ ID NO: 42) | | | | | 169: 5089-97 |
| GILNVSAVDKSTGK | HLA- | human | hspa8 (484-497) | h | Lippolis et al. |
| (SEQ ID NO: 43) | DRB*0401 | | | | (2002), J. Imm., |
| | | | | | 169: 5089-97 |
| GILNVSAVDKSTGKE | HLA- | human | hspa8 (484-498) | h | Dengjel et al., |
| (SEQ ID NO: 44) | DRB1*0401/ | | | | (2005), PNAS, |
| | DRB4*0101 | | | | 102: 7922-7927 |
| | | | | | tbl 1 |
| CNEIINWLDKNQ | HLA- | human | hspa8 (574-585) | h | Friede et al. |
| (SEQ ID NO: 45) | DR4Dw10 | | | | (1996), BBA, |
| | | | | | 1316: 85-101 |
| ISWLDKNQTAEKEEFE | HLA-DQ8 | human | hspa8 (578-593) | m | Suri et al., |
| (SEQ ID NO: 46) | (transgenic in | | | | (2005), J. Clin. |
| | NOD!) | | | | Invest., |
| | | | | | 115: 2268-2276 |
| | | | | | (suppl) |
| YGSGGPPPTGEEDTS | I-Ag7 | mouse | hspa5 (636-655) | m | Suri et al., |
| EKDEL | | | | | (2005), J. Clin. |
| (SEQ ID NO: 47) | | | | | Invest., |
| | | | | | 115: 2268-2276 |
| | | | | | (suppl) |

($)Species from which the HSP70 protein originates: m = mouse; h = human

1. A method for treating or preventing an Inflammatory Bowel Disease (IBD) comprising administering a composition comprising a full-length heat shock 70 protein (HSP70), a fragment thereof, or both to an animal subject.

2. The method according to claim 1, wherein the IBD is selected from the group consisting of: Crohn's Disease, Granulomatous Colitis, Lymphocyte Colitis, Collagenous Colitis, Ulcerative Colitis, and Coeliac Disease.

3. The method according to claim 2, wherein the IBD is Ulcerative Colitis.

4. The method according to claim 3 wherein the full-length HSP70 is a non-human HSP70 protein.

5. The method according to claim 1, wherein the HSP70 is mammalian HSP70 or bacterial HSP70.

6. The method according to claim 1, wherein the HSP70 comprises at least 50% amino acid sequence identity to SEQ ID NO: 55.

7. The method according to claim 1, wherein the HSP70 fragment comprises or consists of any one of SEQ ID NOS: 1-5 and SEQ ID NOS: 17-47.

8. The method according to claim 1, wherein the composition comprises at least one mammalian HSP70 protein or fragment thereof and at least one bacterial HSP70 protein or fragment thereof.

9. The method according to claim 1, wherein the animal subject is a farm animal or companion animal species.

10. The method according to claim 1 wherein the animal subject is a human subject.

11. A pharmaceutical composition for the treatment or prevention of an IBD comprising a full-length HSP70 protein, a fragment thereof, or both, wherein the HSP70 is mammalian or bacterial HSP70.

12. The composition according to claim 11, wherein the mammalian HSP70 protein is a non-human mammalian HSP70.

13. The composition according to claim 11, comprising at least one mammalian HSP70 protein or fragment thereof and at least one bacterial HSP70 protein or fragment thereof.

14. The composition according to claim 11, wherein the HSP70 comprises at least 70% amino acid sequence identity to SEQ ID NO: 55.

15. The composition according to claim 11, wherein the fragment comprises any one of SEQ ID NO: 1-5 and SEQ ID NO: 17-47.

16. A pharmaceutical composition for the treatment or prevention of IBD comprising at least one fragment of a HSP70 protein, wherein HSP70 protein is a mammalian or a bacterial HSP70 protein and wherein the fragment is between 10 and 30 contiguous amino acids in length.

17. A method for identifying a fragment of a HSP70 protein suitable for treating or preventing an IBD, comprising:
(a) obtaining one or more HSP70-derived peptides;
(b) optionally determining whether the HSP70-derived peptides are capable of binding an MHC class II molecule;
(c) testing the capacity of the peptide(s) to induce peptide specific T-cells that are cross-reactive with a homologous self-peptide of a mammal in a cross-reactivity assay; and
(d) selecting those peptides which do show cross-reactivity.

18. The method according to claim 17 further comprising:
(e) administering a composition comprising those peptides selected from (d) in an animal model of an IBD disease to determine the in vivo protective activity; and
(f) comparing disease development and/or symptoms between treated and control animals.
